(19) United States
(12) Patent Application Publication
(10) Pub. No.: US 2024/0196940 A1
(43) Pub. Date: Jun. 20, 2024

(54) COLOR-FAST EDIBLE COMPOSITIONS AND METHODS OF PRODUCING SAME

(71) Applicant: Triton Algae Innovations, Ltd., San Diego, CA (US)

(72) Inventors: Susanna Rose KAHN, San Diego, CA (US); Xun WANG, San Diego, CA (US)

(21) Appl. No.: 18/556,018

(22) PCT Filed: May 2, 2022
(86) PCT No.: PCT/US22/27277

§ 371 Date: Oct. 18, 2023

Related U.S. Application Data
(60) Provisional application No. 63/183,174, filed on May 3, 2021.

(51) Int. Cl. A23L 5/46; A23J 3/16; A23J 3/18; A23J 3/22; A23J 3/26; A23L 5/10

(52) U.S. Cl. CPC A23L 5/46; A23J 3/16; A23J 3/18; A23J 3/227; A23J 3/26; A23L 5/10

(57) ABSTRACT

Provided herein are color-fast edible compositions and methods of making the same. In some cases, the color-fast edible compositions comprise a comprising an exogenous protein component and a biomass comprising cells of a pigmented algae. The pigmented algal cells may be physically associated with the exogenous protein component such that the pigmented algal cells confer a pigmentation to the edible compositions and are substantially dissociable from the exogenous protein component. Further provided herein are food products (e.g., meat substitutes, fish or shellfish substitutes) comprising the color-fast edible compositions of the disclosure.

### CROSS-REFERENCED TO RELATED APPLICATION

This application claims priority benefit to U.S. provisional application Ser. No. 63/183,174, filed May 3, 2021, which application is incorporated herein by reference in its entirety.

### BACKGROUND

The increasing popularity of plant and other non-animal product-based diets has led to a demand for meat and seafood substitutes. Meat and seafood substitutes often have a pigment added to the product (e.g., a red coloring) such that the product more closely resembles the natural meat or seafood product for which they are substituted. In some instances, the added pigment may diffuse or leach from the meat or seafood substitute, for example, when the meat or seafood substitute is in contact with a liquid (e.g., water), or during the cooking process. Leaching of the pigment may result in a loss of color to the food substitute, a loss of taste of the food substitute, or both. Additionally, leaching of the pigment may result in a loss of aesthetic appeal of the food product.

### SUMMARY

There is an unmet need for meat and seafood substitutes that retain their pigmentation and do not leach pigment from the product. The present disclosure provides color-fast edible compositions, food products comprising the color-fast edible compositions, and methods of making the same, which meet this unmet need.

In one aspect, an edible composition is provided comprising: a color-fast mixture comprising an exogenous protein component physically associated with a biomass comprising cells of a Chlamydomonas algae, wherein the exogenous protein component comprises at least about 20% protein (dry weight), such that the biomass confers a red or red-brown pigmentation to the exogenous protein component that is substantially non-dissociable from the exogenous protein component.

In some embodiments, the disclosure provides a color-fast extruded edible composition comprising an exogenous protein component physically associated with a biomass comprising pigmented algae cells, wherein the biomass confers a pigmentation to the mixture that is substantially non-dissociable from the color-fast extruded edible composition. In some cases, the pigmented algae cells contain a red or red-brown pigmentation. In some cases, the pigmented algae cells are Chlamydomonas algae cells. In some cases, the color-fast extruded edible composition comprises at least about 1%, at least about 2%, at least about 5%, at least about 10%, at least about 15% or at least about 20% protein by dry weight of the extruded mixture. The color-fast extruded edible composition can be in a puff form or in a fibrous form.

In some cases, the exogenous protein component comprises a gellable protein. In some cases, the gellable protein is in a gel form and the red or red-brown pigmentation is physically associated with the outside surface of the gel form or as a discontinuous phase trapped within the gel form. In some cases, the gellable protein is in a gel form and is a textured protein. In some cases, the color-fast mixture has been heat-treated. In some cases, the protein comprises a plant protein. In some cases, the plant protein is selected from the group consisting of: a cereal grain protein, a legume protein, a pulse protein, a tree nut protein, a ground nut protein, a seed protein, a tuber protein, a leaf protein, an algae protein, a microalgae protein, a wheat protein, a soy protein, a nut protein, a pea protein, and any combination thereof. In some cases, the protein comprises a fungal protein. In some cases, the fungal protein is selected from the group consisting of: a mushroom protein, a yeast protein, a mycoprotein, and any combination thereof. In some cases, the protein comprises an animal protein. In some cases, the animal protein is selected from the group consisting of: a mammalian protein, an avian protein, a fish protein, a seafood protein, and any combination thereof. In some cases, the Chlamydomonas algae is of the species Chlamydomonas reinhardtii. In some cases, the Chlamydomonas algae comprises a pigment, and wherein the pigment confers the red or red-brown pigmentation to the exogenous protein component. In some cases, the pigment is selected from the group consisting of: heme, protoporphyrin IX, and both heme and protoporphyrin IX. In some cases, a substantial proportion of the red or red-brown pigmentation remains associated with the exogenous protein component when the edible composition is in contact with an aqueous solution (e.g., a solution of 1% NaCl at 25° C. for at least 1 minute). In some cases, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the red or red-brown pigmentation remains associated with the exogenous protein component when the edible composition is in contact with an aqueous solution (e.g., a solution of 1% NaCl at 25° C. for at least 1 minute). In some cases, a substantial proportion of the red or red-brown pigmentation does not enter a liquid phase when the edible composition is immersed in a liquid (e.g., when immersed in an oil for at least 1 minute at 25° C.). In some cases, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the red or red-brown pigmentation does not enter a liquid phase when the edible composition is immersed in a liquid (e.g., when immersed in an oil for at least 1 minute at 25° C.). In some cases, a substantial proportion of the red or red-brown pigmentation does not leach or diffuse into a solid or gel phase when the solid or gel phase is in physical proximity (e.g., for at least 1 minute at 25° C.) with the edible composition. In some cases, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the red or red-brown pigmentation does not leach or diffuse into a solid or gel phase when the solid or gel phase is in physical proximity (e.g., for at least 1 minute at 25° C.) with the edible composition. In some cases, the red or red-brown pigmentation is substantially water insoluble (e.g., at 25° C.). In some cases, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the red or red-brown pigmentation is substantially water insoluble (e.g., at 25° C.). In some cases, at least about 0.1%, at least about 0.5%, at least about 1%, at least about 1.5%, at least about 2%, at least about 3%, at least about 4%, or at least about 5% of the edible composition comprises the biomass. In some cases, less than about 0.1%, less than about 0.5%, less than about 1%, less than about 1.5%, less than about 2%, less than about 3%, less than about 4%, or less than about 5% of the edible composition comprises the biomass. In some cases, the edible composition further comprises a salt. In some cases, the color-fast mixture is comprised of substantially aligned fibers. In some cases, the exogenous protein component is comprised of substantially aligned fibers. In some cases, the edible composition further comprises one or more flavorings. In some cases, the one or more flavorings confers a fish-like flavor to the edible composition. In some cases, the edible composition has a flake-like shape, a flake-like texture, or both.

In another aspect, a seafood analog is provided comprising the edible composition of any one of the preceding. In some cases, the seafood analog is a shellfish analog. In some cases, the shellfish analog is selected from the group consisting of: a shrimp analog, a crab analog, a scallop analog, and a lobster analog. In some cases, the seafood analog is a fish analog. In some cases, the fish analog is selected from the group consisting of a tuna analog, a salmon analog, and a cod analog.

In another aspect, a meat analog is provided comprising the edible composition of any one of the preceding. In some cases, the meat analog is selected from the group consisting of a beef analog, a turkey analog, a duck analog, an ostrich analog, a dark-meat poultry analog, and a pork analog. In some cases, a meat analog is a pulled pork analog or barbecue pork analog.

In yet another aspect, a food product is provided, comprising: (a) a first component comprising an exogenous protein physically associated with pigmented algal cells, in contact with (b) a second component which substantially lacks pigmented algal cells. In some cases, the second component is selected from the group consisting of: a liquid, a gel, a foam, and a solid. In some cases, the exogenous protein comprises a plant protein. In some cases, the plant protein is selected from the group consisting of: a cereal grain protein, a legume protein, a pulse protein, a tree nut protein, a ground nut protein, a seed protein, a tuber protein, a leaf protein, an algae protein, a microalgae protein, a wheat protein, a soy protein, a nut protein, a pea protein, and any combination thereof. In some cases, the exogenous protein comprises a fungal protein. In some cases, the fungal protein is selected from the group consisting of: a mushroom protein, a yeast protein, a mycoprotein, and any combination thereof. In some cases, the exogenous protein comprises an animal protein. In some cases, the animal protein is selected from the group consisting of: a mammalian protein, an avian protein, a fish protein, a seafood protein, and any combination thereof. In some cases, the exogenous protein comprises at least about 20%, at least about 25%, at least about 30%, at least about 40%, at least about 50%, at least about 60%, at least about 70%, at least about 80%, at least about 90%, or at least about 95% protein. In some cases, the pigmented algal cells are microalgae cells. In some cases, the microalgae cells are of the genus Chlamydomonas. In some cases, the pigmented algal cells comprise a pigment that confers a pigmentation to the first component. In some cases, the pigment is selected from the group consisting of: heme, protoporphyrin IX, and both heme and protoporphyrin IX. In some cases, the pigmented algal cells comprise a mutation in one or more genes in a pathway for chlorophyll synthesis, protoporphyrinogen IX synthesis, or heme synthesis. In some cases, the pigmented algal cells comprise a mutation in one or more genes selected from the group consisting of: CHLD, CHLI1, CHLI2, CHLH1, ChlB, ChlL, ChlN, protoporphyrinogen IX oxidase, ferrocheletase, protochlorophyllide oxidoreductase, and magnesium chelatase. In some cases, the pigmented algal cells comprise a mutation in one or more genes that results in a phenotype selected from the group consisting of: upregulated expression of protoporphyrinogen IX oxidase, upregulated expression of ferrocheletase, reduction or elimination of expression of light independent protochlorophyllide oxidoreductase, and a deficiency in the expression of magnesium chelatase. In some cases, the pigmentation is a red or a red-brown color. In some cases, the food product is a meat substitute or a seafood substitute. In some cases, the meat substitute is a beef substitute or a pork substitute. In some cases, the seafood substitute is a shellfish substitute. In some cases, the shellfish substitute is selected from the group consisting of: a shrimp substitute, a crab substitute, a scallop substitute, and a lobster substitute. In some cases, the seafood substitute is a fish substitute. In some cases, the fish substitute is selected from the group consisting of: a tuna substitute, a salmon substitute, and a cod substitute. In some cases, the food product is selected from the group consisting of: a burger, a patty, a hot dog, a meatball, a ground meat, a ground meat product, a meat sauce, a shredded meat, a nugget, a fish ball, a chopped fish, a shredded fish, a fish salad, sushi, sashimi, and a filet. In some cases, the pigmented algal cells comprise at least about 0.1%, at least about 0.5%, at least about 1%, at least about 2%, at least about 3%, at least about 4%, or at least about 5% by weight of the first component. In some cases, the food product further comprises a salt. In some cases, the second component comprises a liquid selected from the group consisting of: a broth, a gravy, a sauce, a soup, a dressing, and a glaze. In some cases, the second component comprises a liquid selected from the group consisting of: water and an aqueous salt solution. In some cases, the second component comprises a liquid selected from the group consisting of: an oil and a fat. In some cases, the second component is a solid selected from the group consisting of: a pasta, a noodle, a bread, a bun, a wrapper, a dough, a coating, a grain, a starch, and an icing. In some cases, the second component is a gel selected from the group consisting of: a mousse, a gummy, a cooked egg, a cake, a bread, a custard, a pie filling, and a flan. In some cases, the food product is selected from the group consisting of: a burger, a sandwich, a soup, a dumpling, a stew, a casserole, a wrap, a salad, a biryani, a paella, a jambalaya, and a meatball in sauce.

In some cases, the second component is a second exogenous protein component. The second exogenous protein component can be a plant protein, a fungal protein, or an animal protein. Certain non-limiting examples of plant, fungal, and animal proteins are provided above and any such protein can be used as a second exogenous protein component.

In some cases, the second component is a second Chlamydomonas algae biomass.

In yet another aspect, a method for making a color-fast edible composition is provided, the method comprising: (a) combining an exogenous protein component comprising at least about 20% protein (dry weight), with a biomass comprising cells of a pigmented algae to produce a mixture; and (b) heating the mixture thereby physically associating the cells of the pigmented algae with the exogenous protein component such that the cells of the pigmented algae confer a pigmentation to the exogenous protein component and the pigmentation is substantially non-dissociable from the exogenous protein component, thereby making the color-fast edible composition.

In some cases, the method further comprises combining the mixture of (a) with at least one additional component prior to or after the heating of (b). In some cases, the at least one additional component is a flavoring component. In some cases, the flavoring component imparts a meat-like flavor, a fish-like flavor, or a shellfish-like flavor to the color-fast edible composition. In some cases, the heating of (b) comprises heating the mixture to a temperature of at least about 80° C. In some cases, the heating of (b) comprises heating the mixture to a temperature of at least about 100° C., at least about 110° C., at least about 120° C., at least about 130° C., at least about 140° C., or at least about 150° C. In some cases, the heating of (b) comprises exposing the mixture to steam. In some cases, the heating of (b) comprises heating the mixture under substantially dry conditions. In some cases, the heating of (b) comprises heating by extrusion. In some cases, the substantially dry conditions comprise heating the mixture in contact with no more than about 20%, no more than about 15%, no more than about 10%, no more than about 5%, no more than about 3%, no more than about 2%, or no more than about 1% humidity. In some cases, the heating of (b) comprises pressure cooking the mixture. In some cases, the heating of (b) comprises boiling the mixture. In some cases, the exogenous protein component comprises at least about 25%, at least about 30%, at least about 40%, at least about 50%, at least about 60%, at least about 70%, at least about 80%, or at about least 90% protein (dry weight). In some cases, the method further comprises washing the color-fast edible composition with an aqueous solution. In some cases, the method further comprises agitating the color-fast edible composition with an aqueous solution. In some cases, the method further comprises combining the color-fast edible composition with a liquid. In some cases, the liquid is selected from the group consisting of: a broth, a soup, a sauce, a gravy, a dressing, and a glaze. In some cases, the liquid is selected from the group consisting of: a water and an aqueous salt solution. In some cases, the liquid is selected from the group consisting of: an oil and a fat. In some cases, the method further comprises combining the edible composition with a solid.

Further embodiments of the disclosure provide a method of making a color-fast extruded edible composition, the method comprising: extruding a mixture comprising an exogenous protein component and pigmented algae cells so as to physically associate the pigmented algae cells with the exogenous protein component to confer a pigmentation to the exogenous protein component, wherein the pigmentation is substantially non-dissociable from the exogenous protein component, thereby making the color-fast extruded edible composition.

In certain such methods, the exogenous protein component comprises at least about 1%, at least about 2%, at least about 5%, at least about 10%, or at least about 15% of protein by dry weight of the color-fast extruded edible composition. In some cases, the exogenous protein component comprises at least about 20%, at least about 25%, at least about 30%, at least about 40%, at least about 50%, at least about 60%, at least about 70%, at least about 80%, or at about least 90% protein by dry weight of the color-fast extruded edible composition.

The color-fast extruded edible compositions of the present disclosure can be incorporated in a variety of different edible compositions, e.g., meat analog (or meat substitute) (e.g., red meat substitute (e.g. beef substitute), white meat substitute (e.g., pork substitute, avian substitute, e.g., poultry substitute (e.g., chicken, turkey); seafood analogs (e.g., tuna, crab, etc.), and the like. Such analogs can be produced based on different concentration of pigmented algae cells (e.g. Chlamydomonas algae cells), flavoring agents, parameters of extruding process, etc.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 demonstrates that heat treatment of a composition comprising an exogenous protein component and pigmented algal cells prevents or reduces leaching of pigment into water.

FIG. 2A depicts a comparison of non-cooked and pressure-cooked compositions of the disclosure in a mixture of water and oil. FIG. 2B depicts a comparison of non-cooked and steam-cooked compositions of the disclosure in a mixture of water and oil.

FIG. 3 depicts a comparison of heat-treated compositions of the disclosure comprising increasing amounts of red algae.

FIG. 4 depicts color-fast compositions according to embodiments of the disclosure.

FIG. 5 depicts color-fast compositions of the disclosure affixed to microscope slides. (WW=without washing and W=washing.)

FIGS. 6A-6B depict microscopy images of color-fast compositions of the disclosure.

FIG. 7 depicts slides prepared for microscopy containing color-fast compositions of the disclosure.

FIGS. 8A-8L depict microscopy images of color-fast compositions of the disclosure prepared using various methods and conditions.

FIGS. 9A-9E depict microscopy images of pigmented algal cells under various conditions.

FIGS. 10A-10G depict microscopy images of pigmented algal cells under various conditions.

FIG. 11 depicts the relative amounts of chlorophyll for a green algae strain, two white algae strains, and a yellow algae strain.

FIG. 12 shows an example of preparing protein puffs containing soy protein and red algae by a method disclosed herein for producing a color-fast extruded edible composition.

FIG. 13 shows a comparison of heat treated and non-heat treated flaked fish products.

### DETAILED DESCRIPTION

Provided herein are color-fast edible compositions (e.g., for use in food products). The color-fast edible compositions generally comprise an exogenous protein component and a biomass comprising cells of a pigmented algae. The color-fast edible compositions may be processed such that the pigmented algal cells become physically associated with the exogenous protein component. In such cases, the pigmented algal cells may be substantially non-dissociable from the exogenous protein component, thereby preventing the pigment from leaching or diffusing from the edible composition (e.g., when the edible composition is in contact with a liquid, when the edible composition is cooked). Further provided herein are food products containing the color-fast edible compositions. In some cases, the food products are substitutes for natural meat or seafood products (e.g., meat substitutes, seafood substitutes). Additionally, provided herein are methods of producing color-fast edible compositions. The methods generally include mixing an exogenous protein component with a biomass comprising cells of a pigmented algae and processing the mixture (e.g., by heat treating) such to produce the color-fast edible composition.

The disclosure herein provides color-fast compositions. The term “color-fast” as used herein refers to a colored or pigmented composition in which the color or pigment does not dissociate or does not substantially dissociate from the composition (e.g., under various conditions). A “color-fast” composition as provided herein may not leach or diffuse or may not substantially leach or substantially diffuse color or pigment from the color-fast composition (e.g., under various conditions). The compositions provided herein may be processed (e.g., by methods provided herein) to generate the color-fast composition.

A “color-fast extruded edible composition” as used herein refers to an extruded composition containing an exogenous protein component and pigmented algae cells, for example, pigmented Chlamydomonas algae cells. Such composition can be in a puff form e.g., a puff protein. Such composition can also be in a fibrous form e.g., a fibrous protein.

The term “puff” as used herein, for example, “puff protein,” “puff form,” or “puff composition,” refers to a color-fast composition containing an exogenous protein, wherein the composition is porous and has crunchy mouthfeel.

The term “fibrous” as used herein, for example, “fibrous protein,” “fibrous form,” or “fibrous composition,” refers to a color-fast composition containing an exogenous protein, wherein the composition has protein fibers, for example, fibers that resemble muscle fibers. Such fibers can be substantially aligned fibers.

In some aspects, a color-fast edible composition of the disclosure comprises an exogenous protein component. The term “protein component” as used herein generally refers to a composition comprising an amount of protein and may or may not include one or more additional components.

The term “exogenous”, when used in conjunction with the term “protein component”, refers to a protein component that is not naturally associated with the pigmented algal cells (e.g., in nature), and is meant to differentiate from natural protein sources that may be associated with algal cells (e.g., in nature). The color-fast compositions provided herein are non-naturally occurring compositions (e.g., do not exist in nature) by virtue of the fact that they contain non-naturally occurring mixtures of an exogenous protein component and pigmented algal cells. Moreover, the color-fast compositions are non-naturally occurring compositions as they generally have undergone one or more processing steps to produce the color-fast compositions (e.g., heat treatment, as provided herein).

The exogenous protein component comprises an amount of a protein. The exogenous protein component may comprise at least about 5% protein (w/w), at least about 10% protein (w/w), at least about 15% protein (w/w), at least about 20% protein (w/w), at least about 25% protein (w/w), at least about 30% protein (w/w), at least about 35% (w/w), at least about 40% (w/w), at least about 45% (w/w), at least about 50% (w/w), at least about 55% (w/w), at least about 60% protein (w/w), at least about 65% protein (w/w), at least about 70% protein (w/w), at least about 75% protein (w/w), at least about 80% protein (w/w), at least about 85% protein (w/w), at least about 90% protein (w/w), at least about 95% protein (w/w), or greater, or at least about 99% protein (w/w). In a preferred example, the exogenous protein component comprises at least about 20% protein (w/w). In some cases, the exogenous protein component is a textured protein or gellable protein and comprises from about 10% to about 60% (solids basis) of the final (wet basis) product. In some cases, the exogenous protein component is a textured protein or gellable protein and comprises from about 15% to about 50% (solids basis) of the final (wet basis) product.

The exogenous protein component may comprise essentially any protein that is suitable for use in an edible composition, preferably a protein that is gellable. In some instances, the protein is a protein gel. In some instances, the gellable protein is in a gel form and is a textured protein. Protein gels can be created when proteins form a cross-linked three-dimensional network, such as through, e.g., hydrophobic interactions, disulfide bonds, hydrogen bonding, and ionic interactions. A gellable protein is a protein (e.g., a protein composition, a protein isolate, a mixture of proteins, or a single type of protein) where the protein molecules are able to associate closely enough with each other for a gel to form. In some instances, the gellable protein is in a gel form. In some instances, the gellable protein is in a gel form and is a textured protein. In some instances, in addition to a gellable protein, the composition herein may include additional non-gellable or non-texture proteins or protein isolates.

In some instances, the protein comprises a plant protein. Plant proteins suitable for use in the edible compositions provided herein include, without limitation, cereal grain proteins (e.g., wheat protein, millet protein, rice protein, barley protein, oat protein, sorghum protein, maize protein), legume and pulse proteins (e.g., pea protein, chickpea protein, lentil protein, bean protein), tree nut proteins (e.g., Brazil nut protein, cashew protein, hazelnut protein, macadamia nut protein, pecan protein, pine nut protein, pistachio nut protein, almond protein, walnut protein), ground nut proteins (e.g., peanut protein), seed proteins, tuber proteins, leaf proteins, algae proteins, microalgae proteins, wheat proteins, soy proteins, nut proteins, and pea proteins. In some instances, the protein comprises a fungal protein. Fungal proteins suitable for use in the edible compositions of the disclosure include, without limitation, mushroom proteins, yeast proteins, and mycoproteins. In some instances, the protein comprises an animal protein. Animal proteins suitable for use in an edible composition of the disclosure include, without limitation, a mammalian protein (e.g., cow, sheep, pig, etc.) (e.g., such as from meat or dairy material), an avian protein (e.g., chicken, turkey, etc.) (e.g., such as from a meat or egg material), a fish protein, and a shellfish protein. In a non-limiting embodiment, the protein is a wheat protein, a textured wheat protein, or a wheat protein isolate.

The protein may be selected based on any number of desired characteristics, including, without limitation, the desired taste of the edible composition, the desired texture of the edible composition, the desired shape of the edible composition, the desired mouthfeel of the edible composition, the desired smell of the edible composition, and the like. In some cases, the protein is a gellable protein or is present in a gel form or a texture form. In some cases, the present disclosure provides vegetarian or vegan compositions, and the protein may be selected accordingly. For example, a vegetarian or vegan composition may comprise a protein sourced from a non-animal (e.g., a plant). Similarly, a vegetarian or vegan composition may comprise an animal protein produced in a non-animal cell (e.g., recombinantly produced in, e.g., bacteria or yeast). The exogenous protein component may comprise a protein that is naturally occurring (e.g., in nature). In other cases, the exogenous protein component may comprise a non-naturally occurring protein (e.g., a protein that does not exist in nature). In some cases, the exogenous protein component may comprise a naturally occurring protein or a non-naturally occurring protein that has been produced by a non-natural production method (e.g., recombinantly produced).

The color-fast edible compositions provided herein further comprise a biomass comprising pigmented algal cells. The pigmented algal cells generally are selected such that they confer a desired pigmentation to the composition. One object of the disclosure is to provide meat and seafood substitutes. In such cases, the pigmented algal cells are selected such that they confer a pigmentation to the composition that resembles or mimics the natural meat or seafood pigmentation. In a preferred embodiment, the pigmented algal cells are selected such that they confer a red or red-brown pigmentation to the composition. The algal cells may naturally produce a pigment (e.g., a red or red-brown pigment). In other cases, the algal cells may be genetically engineered or altered to produce or overproduce a pigment (e.g., a red or red-brown pigment). The algal cells generally produce enough pigment such that they confer a pigmentation (e.g., red or red-brown) to the composition. In some instances, the algal cells may be selected such that they do not produce, or produce in low amounts, pigments that confer undesirable colors to the composition. In some instances, the algal cells may naturally produce an undesired pigment but have been genetically altered (e.g., mutated) such that the cell no longer produces, or produces less of, the undesired pigment. In one such example, the algal cells may produce chlorophyll (green pigment) but in an amount that is low enough to not confer a green pigmentation to the composition.

In a preferred embodiment, the pigmented algal cells confer a red or red-brown pigmentation to the composition. Any algae (e.g., red algae) that produces a red or red-brown pigment is suitable for use in the compositions provided herein. In a non-limiting example, the algae are of the genus Chlamydomonas. In some aspects, the algae are Porphyridium sp., Porphyridium crentum, Porphyridium aerugineum, Haematococcus pluvialis, Flintiella sanguinaria, or a Chlamydomonas sp. In another non-limiting example, the algae are of the species Chlamydomonas reinhardtii. In some cases, the algal cells naturally produce, or have been genetically engineered to produce or over-produce heme. Additionally or alternatively, the algal cells naturally produce, or have been genetically engineered to produce or over-produce protoporphyrin IX. Although specific examples have been given, the disclosure is not limited to such, and the algal cells may produce any pigment, including a red or red-brown pigment, or green pigment. Advantageously, the algal cells provide a natural pigment to the color-fast composition (rather than a synthetic dye or additive).

In various aspects, the pigmented algal cells may comprise a mutation in one or more genes in a pathway for one or more of chlorophyll synthesis, protoporphyrinogen IX synthesis, or heme synthesis. In some instances, the pigmented algal cells comprise a mutation in one or more genes selected from the group consisting of: CHLD, CHLI1, CHLI2, CHLH1, ChlB, ChlL, ChlN, protoporphyrinogen IX oxidase, ferrocheletase, protochlorophyllide oxidoreductase, and magnesium chelatase. In some instances, the pigmented algal cells comprise a mutation in one or more genes that results in a phenotype selected from the group consisting of: upregulated expression of protoporphyrinogen IX oxidase, upregulated expression of ferrocheletase, reduction or elimination of expression of light independent protochlorophyllide oxidoreductase, and a deficiency in the expression of magnesium chelatase.

The color-fast edible compositions provided herein generally contain an amount of biomass (e.g., comprising the pigmented algal cells) that is sufficient to confer a desired pigmentation to the composition. In some instances, at least about 0.1%, at least about 0.5%, at least about 1%, at least about 1.5%, at least about 2%, at least about 3%, at least about 4%, or at least about 5% of the color-fast composition comprises the biomass. In other instances, less than about 0.1%, less than about 0.5%, less than about 1%, less than about 1.5%, less than about 2%, less than about 3%, less than about 4%, or less than about 5% of the color-fast composition comprises the biomass.

Generally, the color-fast edible compositions of the disclosure are processed (e.g., by the methods provided herein) to produce the color-fast compositions. In some cases, the color-fast compositions comprise a mixture (of the exogenous protein component and the pigmented algal cells) that has been heat treated (e.g., subjected to heat) to produce the color-fast compositions. The processing (e.g., heat treatment) may result in the physical association of the pigmented algal cells with the exogenous protein component. Without wishing to be bound by theory, it is thought that the processing (e.g., heat treatment) results in the physical association of the pigmented algal cells with the outside surface of the exogenous protein component, such as the outside surface of a protein gel or as a discontinuous phase trapped within a continuous protein gel or other protein matrix. The physical association of the pigmented algal cells with the exogenous protein component results in a color-fast composition (e.g., as disclosed herein).

In one aspect, a substantial proportion of the pigmentation (e.g., red or red-brown pigmentation) of the color-fast composition remains associated with the exogenous protein component when the color-fast composition is in contact with an aqueous solution (e.g., a solution of 1% NaCl at 25° C. for at least 1 minute) or a liquid. For example, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the pigmentation (e.g., red or red-brown pigmentation) of the color-fast composition remains associated with the exogenous protein component when the color-fast composition is in contact with an aqueous solution (e.g., a solution of 1% NaCl at 25° C. for at least 1 minute) or a liquid.

In another aspect, a substantial proportion of the pigmentation (e.g., red or red-brown pigmentation) of the color-fast composition does not enter the liquid phase when the color-fast composition (e.g., of the disclosure) is immersed in a liquid (e.g., when immersed in an oil for at least 1 minute at 25° C.). For example, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the pigmentation (e.g., red or red-brown pigmentation) of the color-fast composition does not enter the liquid phase when the color-fast composition is immersed in a liquid (e.g., when immersed in an oil for at least 1 minute at 25° C.).

In another aspect, a substantial proportion of the pigmentation (e.g., red or red-brown pigmentation) of the color-fast composition does not leach into a solid phase or a gel phase when the solid phase or the gel phase is in physical proximity (e.g., for at least 1 minute at 25° C.) with the color-fast composition. For example, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the pigmentation (e.g., red or red-brown pigmentation) of the color-fast composition does not leach into a solid phase or a gel phase when the solid phase or the gel phase is in physical proximity (e.g., for at least 1 minute at 25° C.) with the color-fast composition.

In another aspect, the pigmentation (e.g., red or red-brown pigmentation) of the color-fast composition is substantially water insoluble (e.g., at 25° C.). For example, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the pigmentation (e.g., red or red-brown pigmentation) of the color-fast composition is substantially water insoluble (e.g., at 25° C.).

In some embodiments, the color-fast composition may include an exogenous protein component where the protein fibers are aligned. In some aspects, the protein fibers of the exogenous protein component are aligned prior to the addition of the pigmented algal cells and to the color-fasting (e.g., as described herein). In some aspects, the pigmented algal cells are combined with the exogenous protein component and then the protein fibers of the mixture are aligned prior to the color-fasting. In some aspects, the color-fast composition exhibits a visible appearance of aligned fibers, such that it resembles a fish, seafood, or meat product that has aligned protein fibers.

In various aspects, the color-fast composition may comprise one or more additional components. In some cases, the color-fast composition comprises one or more flavorings. Generally, the one or more flavorings are selected to impart a flavor to the color-fast composition such that the composition mimics a flavor of a natural meat or seafood substitute. For example, when the color-fast composition is used in a seafood substitute, the one or more flavorings may be selected to impart a fish-like flavor to the composition. Similarly, when the color-fast compositions are used in a meat substitute, the one or more flavorings are selected to impart a meat-like (e.g., beef) flavor to the composition. Additional ingredients may be added to the color-fast composition depending on the intended use of the composition. For example, additional ingredients may be added to impart additional characteristics to the composition (e.g., that mimic or resemble a seafood or meat product). Such additional ingredients include ingredients that impart an aroma or smell that mimics or resembles the aroma or smell of a seafood or meat product, ingredients that impart a texture that mimics or resembles the texture of a seafood or meat product, ingredients that impart a mouthfeel that mimics or resembles the mouthfeel of a natural seafood or meat product, and the like.

Further provided herein are food products comprising the color-fast edible compositions (e.g., as described herein). The food products may be substitutes or analogs of natural meat or seafood products. The terms “substitute” and “analog” are used interchangeably throughout, and, when used in the context of food products, generally refer to food products that mimic or resemble one or more characteristics (e.g., color, smell, taste, texture, shape, and the like) of natural meat or seafood products.

In various aspects, the food product is a seafood substitute. The seafood substitute may be a shellfish substitute. The shellfish substitute may be a shrimp substitute, a crab substitute, a scallop substitute, a prawn substitute, a squid substitute, an octopus substitute, a crawfish substitute, or a lobster substitute. The seafood substitute may be a fish substitute. The fish substitute may be a tuna substitute, a salmon substitute, a pollock substitute, or a cod substitute. In a non-limiting embodiment, the seafood substitute is a tuna substitute. The seafood substitute generally includes a color-fast composition of the disclosure and may additionally comprise one or more additional ingredients that confer or impart to the seafood substitute one or more characteristics of a natural seafood product. In preferred embodiments, the seafood substitute, or a portion thereof, is a substitute for a natural seafood product that has a red or red-brown color. In such cases, the seafood substitute comprises a color-fast composition that has a red or red-brown pigmentation (e.g., containing pigmented algal cells that produce a red or red-brown pigmentation).

In various aspects, the food product is a meat substitute. The meat substitute may be a beef substitute or a pork substitute. The meat substitute generally includes a color-fast composition of the disclosure and may additionally comprise one or more additional ingredients that confer or impart to the meat substitute one or more characteristics of a natural meat product. In preferred embodiments, the meat substitute, or a portion thereof, is a substitute for a natural meat product that has a red or red-brown color. In such cases, the meat substitute comprises a color-fast composition that has a red or red-brown pigmentation (e.g., containing pigmented algal cells that produce a red or red-brown pigmentation).

Further provided herein are food products comprising a color-fast composition (e.g., of the disclosure) comprising an exogenous protein component physically associated with pigmented algal cells, in contact with a second component which substantially lacks pigmented algal cells. Put another way, the food products comprise a color-fast composition in contact with an additional component (e.g., a solid, a gel, a liquid, a foam, and the like) whereby the pigmented algal cells do not leach or dissociate from the color-fast composition into the additional component. The component in contact with the color-fast composition may be in any phase, including, a solid, a liquid, a gel, and a foam. In some cases, the component in contact with the color-fast composition is a liquid, such as, without limitation, a broth, a gravy, a sauce, a soup, a dressing, a glaze, water, an aqueous solution (e.g., aqueous salt solution), an oil, or a fat. In some cases, the component in contact with the color-fast composition is a solid, such as, without limitation, a pasta, a noodle, a bread, a bun, a wrapper, a dough, a coating, a grain, a starch, or an icing. In some cases, the component in contact with the color-fast composition is a gel, such as, without limitation, a mousse, a batter, an emulsion, a gummy, a cooked egg, a cake, a bread, a custard, a pie filling, or a flan.

By way of example only, the food product may be a dumpling containing a dough-based wrapper and a filling containing a meat substitute (e.g., containing a color-fast composition of the disclosure). The color-fast composition may comprise pigmented algal cells which confer a red or red-brown pigmentation to the meat substitute (including one or more ingredients which confer or impart one or more meat-like characteristics to the meat substitute). The dumpling filling may further comprise one or more additional ingredients (e.g., onions, ginger, garlic, vegetables, and the like) in contact with the color-fast composition. The dumpling may further comprise a dumpling wrapper in contact with the color-fast composition. In this example, the red or red-brown pigmentation is physically associated with the color-fast composition such that the red or red-brown pigmentation does not leach or dissociate into the additional ingredients or into the dumpling wrapper (e.g., during preparation, during storage, during cooking, and the like).

By way of example only, the food product may be a dumpling containing a starch-based wrapper and filling containing a meat substitute (e.g., containing a color-fast composition of the disclosure). In some instances, the dumpling filling substitute may comprise a pork substitute, a beef substitute, a lamb substitute, a chicken substitute, a turkey substitute, a fish substitute, or a shellfish substitute. The dumpling filling may further comprise one or more additional ingredients or flavorings (e.g., onions, ginger, garlic, green onions, miso broth concentrate, white pepper, and the like) in contact with the color-fast composition. The dumpling may further comprise a starch-based dumpling wrapper in contact with the color-fast composition. In some embodiments, the dumpling wrapper may comprise wheat, potato, corn, rice, or tapioca. In some embodiments, the dumpling may be baked, steamed, fried, boiled, or roasted.

By way of example only, the food product may be a shelf-stable flaked tuna fish substitute (e.g., containing a color-fast composition of the disclosure). In some instances, the flaked fish substitute may be a salmon substitute, a cod substitute, a mackerel substitute, a trout substitute, a rainbow trout substitute, an anchovy substitute, or herring substitute. In some instances, the flaked fish substitute may be a shellfish substitute, a shrimp substitute, a crab substitute, a scallop substitute, a prawn substitute, a squid substitute, an octopus substitute, a crawfish substitute, or a lobster substitute.

In some aspects, the food product is a flaked fish substitute and comprises a color-fast composition that includes algal cells which confer a red or red-brown pigmentation to the product. In some aspects, the flaked tuna fish substitute includes about 0.3% to about 0.4% (w/w) wet weight of algal biomass to total product. In some instances, the color-fast composition may comprise pigmented algal cells which confer a red or red-brown pigmentation to the flaked fish substitute at a concentration of at least about 0.1% (w/w), at least about 0.5% (w/w), at least about 1% (w/w), at least about 2% (w/w), at least about 3% (w/w), at least about 4% (w/w), or at least about 5% (w/w). The flaked fish substitute may further comprise a liquid in contact with the color-fast composition. In some aspects, the liquid is water. In some instances, the liquid is one or more liquids (e.g., water, oil, acid, emulsion). In some embodiments, the red or red-brown pigmentation is physically associated with the color-fast composition such that the red or red-brown pigmentation does not leach or dissociate into the liquid in contact with the color-fast composition (e.g., during preparation, during storage, during cooking, and the like). In some aspects, the flaked tuna fish substitute remains stable for 6 months, e.g., such that the product does not become contaminated by microbial contamination. In some instances, the flaked tuna fish substitute remains stable for at least 9 months, at least one year, at least two years, or at least three years. In some aspects, the flaked tuna fish substitute may further comprise one or more additional ingredients (e.g., wheat starch, soy protein isolate, xanthan gum, methylcellulose, hydroxypropylmethylcellulose, palm oil, algal oil, coconut oil, seaweed, pea protein, faba bean protein, wheat protein, canola oil, lentil protein, natural flavors, sunflower oil, sea salt, gum arabic, red algae, yeast extract, pea protein isolate lecithin and spice). In some instances, the flaked fish substitute may further comprise additional ingredients (e.g., stabilizers, emulsifiers, antioxidants, flavorants, and the like). The flaked fish substitute may further comprise aligned protein fibers. In some aspects, the protein fibers are textured wheat protein. In some instances, the protein fibers are plant, fungal, or animal proteins. In some embodiments, the flaked fish substitute comprises aligned protein fibers, such as, e.g., cut across the long edge of the fibers producing flakes. In some instances, the aligned protein fibers may be cut to produce chunks, shavings, or shreds.

The food product may be a meat substitute, such as, without limitation, a beef substitute, a turkey substitute, an ostrich meat substitute, or a pork substitute. The food product may be a seafood substitute, such as a fish substitute or a shellfish substitute. The fish substitute may be, without limitation, a tuna substitute, a salmon substitute, a pollock substitute, or a cod substitute. The shellfish substitute may be, without limitation, a shrimp substitute, a crab substitute, a scallop substitute, a prawn substitute, a squid substitute, an octopus substitute, a crawfish substitute, or a lobster substitute.

The food products may comprise a meat substitute or a seafood substitute (e.g., comprising a color-fast composition of the disclosure). The food product may be, without limitation, a burger, a patty, a hot dog, a meatball, a ground meat, a ground meat product, a meat sauce, a shredded meat, a nugget, a ground fish, a ground fish product, a fish ball, a fish cake, fish stick, fish paste, a chopped fish, a shredded fish, a fish salad, sushi, sashimi, a filet, surimi, a crab stick, a crab cake, a crab ball, a shrimp stick, a shrimp cake, a shrimp ball, shrimp paste, a squid ball, shredded squid, an octopus ball, or a crawfish ball. The food product may be, without limitation, a burger, a sandwich, a soup, a dumpling, a stew, a casserole, a wrap, a salad, a biryani, a paella, a jambalaya, or a meatball in sauce.

The food products provided herein (e.g., comprising a color-fast composition of the disclosure) may be shelf-stable for an extended period of time. For example, the food products provided herein may be shelf-stable for at least 6 months (e.g., at room temperature; e.g., for at least 6 months, at least 9 months, at least 1 year, etc.). Advantageously, the food products provided herein (e.g., comprising a color-fast composition of the disclosure) may not exhibit leaching of pigment from the color-fast composition into an additional component in contact with the color-fast composition over an extended period of time (e.g., for at least 6 months, at least 9 months, at least 1 year, etc.).

Further provided herein are methods of making color-fast edible compositions of the disclosure. In some aspects, the methods comprise combining an exogenous protein component with a biomass comprising cells of a pigmented algae to produce a mixture.

The exogenous protein component may comprise at least about 5% protein (w/w), at least about 10% protein (w/w), at least about 15% protein (w/w), at least about 20% protein (w/w), at least about 25% protein (w/w), at least about 30% protein (w/w), at least about 35% (w/w), at least about 40% (w/w), at least about 45% (w/w), at least about 50% (w/w), at least about 55% (w/w), at least about 60% protein (w/w), at least about 65% protein (w/w), at least about 70% protein (w/w), at least about 75% protein (w/w), at least about 80% protein (w/w), at least about 85% protein (w/w), at least about 90% protein (w/w), at least about 95% protein (w/w), or greater, or at least about 99% protein (w/w). In a preferred example, the exogenous protein component comprises at least about 20% protein (w/w). In some cases, the exogenous protein component is a textured protein or gellable protein and comprises from about 10% to about 60% (solids basis) of the final (wet basis) product. In some cases, the exogenous protein component is a textured protein or gellable protein and comprises from about 15% to about 50% (solids basis) of the final (wet basis) product.

The exogenous protein component may comprise any protein described herein. For example, the protein may be a plant protein (e.g., cereal grain proteins, such as a wheat protein, a barley protein, a corn protein, a rice protein, legume and pulse proteins, tree nut proteins, ground nut proteins, seed proteins, tuber proteins, leaf proteins, algae proteins, microalgae proteins, wheat proteins, soy proteins, nut proteins, pea proteins); a fungal protein (e.g., mushroom proteins, yeast proteins, mycoproteins) or animal proteins (mammalian proteins, avian proteins, fish proteins, shellfish proteins). In a non-limiting embodiment, the protein is a wheat protein or a wheat protein isolate.

The pigmented algal cells can be any pigmented algal cell that confers a desired pigmentation to the composition. The algal cells may naturally produce a pigment (e.g., a red or red-brown pigment). In other cases, the algal cells may be genetically engineered or altered to produce or overproduce a pigment (e.g., a red or red-brown pigment). The algal cells generally are added to the mixture in an amount such that they confer a desired level of pigmentation to the composition.

In a preferred embodiment, the pigmented algal cells confer a red or red-brown pigmentation to the composition. Any algae (e.g., red algae) that produces a red or red-brown pigment is suitable for use in the methods provided herein. In some aspects, the algae are Porphyridium sp., Porphyridium crentum, Porphyridium aerugineum, Haematococcus pluvialis, Flintiella sanguinaria, or a Chlamydomonas sp. In a non-limiting example, the algae are of the genus Chlamydomonas. In another non-limiting example, the algae are of the species Chlamydomonas reinhardtii. In some cases, the algal cells naturally produce, or have been evolved to produce or over-produce heme. Additionally or alternatively, the algal cells naturally produce, or have been genetically engineered to produce or over-produce protoporphyrin IX. Although specific examples have been given, the disclosure is not limited to such, and the algal cells may produce any red or red-brown pigment. Advantageously, the algal cells provide a natural pigment to the color-fast composition (rather than a synthetic dye or additive).

The methods generally involve adding an amount of biomass comprising pigmented algal cells to the mixture that is sufficient to confer a desired pigmentation to the composition. In some instances, at least about 0.1%, at least about 0.5%, at least about 1%, at least about 1.5%, at least about 2%, at least about 3%, at least about 4%, or at least about 5% (by weight) of the color-fast composition comprises the biomass. In other instances, less than about 0.1%, less than about 0.5%, less than about 1%, less than about 1.5%, less than about 2%, less than about 3%, less than about 4%, or less than about 5% (by weight) of the color-fast composition comprises the biomass. In some aspects, the composition is a darker meat alternative and the amount of biomass comprising pigmented algal cells is about 0.5% or more than about 0.5%. In some aspects, the composition is a lighter meat alternative or a seafood alternative, such as a fish or a tuna alternative, and the amount of biomass comprising pigmented algal cells is about 0.5% or up to about 0.5% or less than about 0.5%.

In various aspects, the methods further comprise heating the mixture to physically associate the pigmented algal cells to the exogenous protein component. In various aspects, the methods involve heating the mixture to a temperature of at least about 80° C. For example, the methods may involve heating the mixture to a temperature of at least about 80° C., at least about 85° C., at least about 90° C., at least about 95° C., at least about 100° C., at least about 105° C., at least about 110° C., at least about 115° C., at least about 120° C., at least about 125° C., at least about 130° C., at least about 135° C., at least about 140° C., at least about 145° C., or at least about 150° C.

In some aspects, the methods involve exposing the mixture (e.g., containing the exogenous protein component and the pigmented algal cells) to steam. In some aspects, the methods involve pressure cooking the mixture (e.g., containing the exogenous protein component and the pigmented algal cells). In some aspects, the methods involve boiling the mixture (e.g., containing the exogenous protein component and the pigmented algal cells). In some aspects, the methods involve heating the mixture (e.g., containing the exogenous protein component and the pigmented algal cells) by microwave, radiant, heat exchangers, or by heating with friction such as in an extruder. In some aspects, the methods involve heating the mixture (e.g., containing the exogenous protein component and the pigmented algal cells) under substantially dry conditions. For example, the methods may involve heating the mixture (e.g., containing the exogenous protein component and the pigmented algal cells) in contact with no more than about 20%, no more than about 15%, no more than about 10%, no more than about 5%, no more than about 3%, no more than about 2%, or no more than about 1% humidity.

Without wishing to be bound by theory, it is thought that the heat treatment physically associates the pigmented algal cells with the exogenous protein component, such that the pigmented algal cells confer a pigmentation (e.g., red or red-brown pigmentation) to the color-fast composition. Without wishing to be bound by theory, It is thought that the processing (e.g., heat treatment) results in the physical association of the pigmented algal cells with the outside surface of the exogenous protein component, such as the outside surface of a protein gel or as a discontinuous phase trapped within a continuous protein gel or other protein matrix. The physical association of the pigmented algal cells with the exogenous protein component results in a color-fast composition. Additionally, it is thought that the heat treatment physically associates the pigmented algal cells with the exogenous protein component such that the pigmented algal cells are substantially non-dissociable from the exogenous protein component. Accordingly, the methods provided herein may result in a color-fast composition wherein the pigmented algal cells confer a pigmentation to the color-fast composition and are substantially non-dissociable from the exogenous protein component.

In various aspects, the methods provided herein produce a color-fast composition (e.g., as described herein) wherein a substantial proportion of the pigmentation (e.g., red or red-brown pigmentation) of the color-fast composition remains associated with the exogenous protein component when the color-fast composition is in contact with an aqueous solution (e.g., a solution of 1% NaCl at 25° C. for at least 1 minute) or a liquid. For example, the methods provided herein produce a color-fast composition (e.g., as described herein) wherein at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the pigmentation (e.g., red or red-brown pigmentation) of the color-fast composition remains associated with the exogenous protein component when the color-fast composition is in contact with an aqueous solution (e.g., a solution of 1% NaCl at 25° C. for at least 1 minute) or a liquid.

In various aspects, the methods provided herein produce a color-fast composition (e.g., as described herein) wherein a substantial proportion of the pigmentation (e.g., red or red-brown pigmentation) of the color-fast composition does not enter the liquid phase when the color-fast composition (e.g., of the disclosure) is immersed in a liquid (e.g., when immersed in an oil for at least 1 minute at 25° C.). For example, the methods provided herein produce a color-fast composition (e.g., as described herein) wherein at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the pigmentation (e.g., red or red-brown pigmentation) of the color-fast composition does not enter the liquid phase when the color-fast composition is immersed in a liquid (e.g., when immersed in an oil for at least 1 minute at 25° C.).

In various aspects, the methods provided herein produce a color-fast composition (e.g., as described herein) wherein a substantial proportion of the pigmentation (e.g., red or red-brown pigmentation) of the color-fast composition does not leach into a solid phase or a gel phase when the solid phase or the gel phase is in physical proximity (e.g., for at least 1 minute at 25° C.) with the color-fast composition. For example, the methods provided herein produce a color-fast composition (e.g., as described herein) wherein at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the pigmentation (e.g., red or red-brown pigmentation) of the color-fast composition does not leach into a solid phase or a gel phase when the solid phase or the gel phase is in physical proximity (e.g., for at least 1 minute at 25° C.) with the color-fast composition.

In various aspects, the methods provided herein produce a color-fast composition (e.g., as described herein) wherein the pigmentation (e.g., red or red-brown pigmentation) of the color-fast composition is substantially water insoluble (e.g., at 25° C.). For example, the methods provided herein produce a color-fast composition (e.g., as described herein) wherein at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the pigmentation (e.g., red or red-brown pigmentation) of the color-fast composition is substantially water insoluble (e.g., at 25° C.).

In various aspects, the methods further involve one or more additional processing steps. In some cases, the one or more additional processing steps involves washing the color-fast edible composition with an aqueous solution. In some cases, the one or more additional processing steps involves agitating the color-fast edible composition with an aqueous solution.

### Color-Fast Extruded Edible Compositions and Methods of Making

In some embodiments, the disclosure provides extrusion methods to produce color-fast extruded edible compositions as well as the compositions produced from such extrusion methods.

Exogenous protein components and pigmented algae used for producing color-fast edible compositions are described above and further herein. These starting materials can also be used in the methods of making color-fast extruded edible compositions.

### Exogenous Protein Component for Use in Color-Fast Extruded Edible Compositions

The exogenous protein component can be a plant protein. The plant protein can be: a cereal grain protein, a legume protein, a pulse protein, a tree nut protein, a ground nut protein, a seed protein, a tuber protein, a leaf protein, an algae protein, a microalgae protein, a wheat protein, a soy protein, a nut protein, a pea protein, and any combination thereof. The plant protein can be a protein concentrate, such as a vegetable protein concentrate, a soy protein concentrate, a wheat protein concentrate or a pea protein concentrate. Such protein concentrates can contain additional components such as carbohydrates, ash, fiber and moisture (water). The exogenous protein component can also be a fungal protein. The fungal protein can be: a mushroom protein, a yeast protein, a mycoprotein, and any combination thereof. The exogenous protein component can also be an animal protein. The animal protein can be: a mammalian protein, an avian protein, a fish protein, a seafood protein, and any combination thereof. Additional details of proteins that can be used as exogenous protein component are described elsewhere in this disclosure and can be used in the color-fast extruded edible compositions and methods of making them as described herein. The exogenous protein component can be a gellable protein. The gellable protein is in a gel form and the red or red-brown pigmentation is physically associated with the outside surface of the gel form or as a discontinuous phase trapped within the gel form. In some cases, the gellable protein is in a gel form and is a textured protein.

In some cases, the exogenous protein component used in the production of the color-fast extruded edible compositions is used in a dried powder form. Thus, the methods of producing the color-fast edible compositions comprise mixing exogenous protein component in dried powder form with pigmented algae cells before extruding such mixture.

### Exogenous Protein Component Concentration

The exogenous protein component can comprise at least about 1% protein, at least about 2%, at least about 5%, at least about 10%, or at least about 15% by dry weight of the color-fast extruded edible composition. In some cases, the protein component comprises at least about 20%, at least about 25%, at least about 30%, at least about 40%, at least about 50%, at least about 60%, at least about 70%, at least about 80%, or at about least 90% by dry weight of the color-fast extruded edible composition.

### Pigmented Algae for Use in Color-Fast Extruded Edible Compositions

As noted above, the pigmented algae cells generally are selected such that they confer a desired pigmentation to the composition. The pigmented algal cells are selected such that they confer a pigmentation to the composition that resembles or mimics the natural meat or seafood pigmentation. In some cases, the pigmented algae cells are selected such that they confer a red or red-brown pigmentation to the composition. In some cases, the pigmented algae cells are selected such that they confer a pink (i.e., lighter red) or pink-red or pink-brown color to the composition. The algal cells may naturally produce a pigment (e.g., a red or red-brown pigment). The algae cells may altered to produce or overproduce a pigment (e.g., a red or red-brown pigment). The algae cells generally produce enough pigment such that they confer a pigmentation (e.g., red or red-brown) to the composition. In some instances, the algae cells may be selected such that they do not produce, or produce in low amounts, pigments that confer undesirable colors to the composition. In some instances, the algal cells may naturally produce an undesired pigment but have been altered (e.g., mutated) such that the cell no longer produces, or produces less of, the undesired pigment. In one such example, the algal cells may produce chlorophyll (green pigment) but in an amount that is low enough to not confer a green pigmentation to the composition.

The pigmented algal cells can confer a red or red-brown pigmentation to the composition. Any algae (e.g., red algae) that produces a red or red-brown pigment is suitable for use in the color-fast extruded edible compositions provided herein. In a non-limiting example, the algae are of the genus Chlamydomonas. In some aspects, the algae are Porphyridium sp., Porphyridium crentum, Porphyridium aerugineum, Haematococcus pluvialis, Flintiella sanguinaria, or a Chlamydomonas sp. In another non-limiting example, the algae are of the species Chlamydomonas reinhardtii. In some cases, the algae cells naturally produce, or have been altered to produce or over-produce heme. Additionally or alternatively, the algae cells naturally produce, or have been altered to produce or over-produce protoporphyrin IX. Although specific examples have been given, the disclosure is not limited to such, and the algae cells may produce any pigment, including a red or red-brown pigment, or green pigment. Advantageously, the algae cells provide a natural pigment to the color-fast composition.

In various aspects, the pigmented algae cells may comprise a mutation in one or more genes in a pathway for one or more of chlorophyll synthesis, protoporphyrinogen IX synthesis, or heme synthesis. In some instances, the pigmented algal cells comprise a mutation in one or more genes selected from the group consisting of: CHLD, CHLI1, CHLI2, CHLH1, ChlB, ChlL, ChlN, protoporphyrinogen IX oxidase, ferrocheletase, protochlorophyllide oxidoreductase, and magnesium chelatase. In some instances, the pigmented algae cells comprise a mutation in one or more genes that results in a phenotype selected from the group consisting of: upregulated expression of protoporphyrinogen IX oxidase, upregulated expression of ferrocheletase, reduction or elimination of expression of light independent protochlorophyllide oxidoreductase, and a deficiency in the expression of magnesium chelatase.

For example, the pigmented algae cells are Chlamydomonas algae cells. The Chlamydomonas algae cells can have, or be modified to have, a red or red-brown pigmentation. The red or red-brown pigmentation of the Chlamydomonas algae cells can be provided by, e.g., heme, protoporphyrin IX, or a combination thereof. In some cases, the cells of the Chlamydomonas algae can be comprise a mutation in one or more genes in a pathway for chlorophyll synthesis, protoporphyrinogen IX synthesis, or heme synthesis.

Certain Non-limiting examples of Chlamydomonas algae cells that can be used in the methods and compositions disclosed herein are described in PCT Publications WO 2020/097363 and WO 2020/097370 and the United States Patent Application Publications US 2020/0332249, US 2021/0401008, and US 2021/0386088, each of which is herein incorporated by reference in its entirety.

In some cases, the pigmented algae cells used in the production of the color-fast extruded edible compositions are used in a dried powder form. Thus, the methods of producing the color-fast edible compositions comprise mixing pigmented algae cells in dried powder form with an exogenous protein before extruding such mixture.

Additional details of pigmented algae are described elsewhere in this disclosure such algae can be used in the color-fast extruded edible compositions and methods of making them described herein.

### Pigmented Algae Concentration

The concentration of the pigmented algae cells can be modified based on the desired use of the color-fast extruded edible composition.

For example, to produce a color-fast extruded edible composition for use in a beef analog, the pigmented algae containing red or red-brown pigmentation can be used at a concentration of 3% to 5% by dry weight of the color-fast extruded edible composition. The color-fast extruded edible composition can then be added to a composition containing fats, flavors, starches, fiber, or binders to produce a beef analog. For example, for use in a beef analog, a color-fast extruded edible composition containing pigmented algae cells containing red or red-brown pigmentation can be made with pigmented algae cells at a concentration of between 3.2% and 4.8%, 3.4% and 4.6%, 3.6% and 4.4%, or 3.8% and 4.2% by dry weight of the color-fast extruded edible composition. In some cases, for use in a beef analog, a color-fast extruded edible composition containing pigmented algae cells containing red or red-brown pigmentation can be made with pigmented algae cells at a concentration of: 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5% by dry weight of the color-fast extruded edible composition. Once the color-fast extruded edible composition containing pigmented algae containing red or red-brown pigmentation is combined with other ingredients to produce a beef analog the resulting composition will contain red algae or red-brown algae at a concentration between 0.2% to 2% of the final weight.

To produce a color-fast extruded edible composition for use in a pork analog, pigmented algae cells containing red or red-brown pigmentation can be used at a concentration of 1% to 3% by dry weight of the color-fast extruded edible composition. For example, for use in a pork analog, a color-fast extruded edible composition containing pigmented algae cells containing red or red-brown pigmentation can be made with pigmented algae cells at a concentration of between 1.2% and 2.8%, 1.4% and 2.6%, 1.6% and 2.4%, or 1.8% and 2.2% by dry weight of the color-fast extruded edible composition. In some cases, for use in a pork analog, a color-fast extruded edible composition containing pigmented algae cells containing red or red-brown pigmentation can be made with pigmented algae cells at a concentration of: 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, or 3% by dry weight of the color-fast extruded edible composition. Once the color-fast extruded edible composition containing pigmented algae containing red or red-brown pigmentation is combined with other ingredients such as fats, flavors, starches, fibers, or binders to produce a pork analog the resulting composition will contain red algae or red-brown algae at a concentration of 0.1% to 1.0% of the final weight.

Moreover, to produce a chicken analog, a color-fast extruded edible composition containing pigmented algae cells containing red or red-brown pigmentation can be used at a concentration of from 0.6% to 1.8% by dry weight of the color-fast extruded edible composition. For example, for use in a chicken analog, a color-fast extruded edible composition containing pigmented algae cells containing red or red-brown pigmentation can be made with pigmented algae cells at a concentration of between 0.7% and 1.7%, 0.8% and 1.6%, 0.9% and 1.5%, 1% and 1.4%, 1.1% and 1.3%, 1.2% and 1.4% by dry weight of the color-fast extruded composition. In some cases, for use in a chicken analog, a color-fast extruded edible composition containing pigmented algae cells containing red or red-brown pigmentation can be made with pigmented algae cells at a concentration of between: 0.6%, 0.7%, 0.8%, 0.9%, %, 1.1%, 1.2%, 1.3%, 1.4%, 1.5%, 1.6%, 1.7%, or 1.8% by dry weight of the color-fast extruded composition. Once the color-fast extruded composition containing pigmented algae containing red or red-brown pigmentation is combined with other ingredients such as fats, flavors, starches, fibers, or binders to produce a chicken analog the resulting composition will contain red algae or red-brown algae at a concentration of 0.05% to 0.5% of the final weight.

To produce a tuna analog, a color-fast extruded edible composition containing pigmented algae cells containing red or red-brown pigmentation can be used at a concentration of from 1.5% to 2.5% by dry weight of the color-fast extruded edible composition. For example, for use in a tuna analog, a color-fast extruded edible composition containing pigmented algae cells containing red or red-brown pigmentation can be made with pigmented algae cells at a concentration of between 1.6% and 2.4%, 1.7% and 2.3%, 1.8% and 2.2%, or 1.9% and 2.1% by dry weight of the color-fast edible composition. In some cases, for use in a tuna analog, a color-fast extruded edible composition containing pigmented algae cells containing red or red-brown pigmentation can be made with pigmented algae cells at a concentration of: 1.5%, 1.6%, 1.7%, 1.8%, 1.9%, 2%, 2.1%, 2.2%, 2.3%, 2.4%, or 2.5% by dry weight of the color-fast extruded edible composition. Once the color-fast extruded edible composition containing pigmented algae containing red or red-brown pigmentation is combined with other ingredients such as fats, flavors, starches, fibers, or binders to produce a tuna analog the resulting composition will contain red algae or red-brown algae at a concentration of 0.075% to 0.75% of the final weight.

### Flavoring Agents

In some cases, the method comprises combining the mixture of (a) with at least one additional component prior to or after extruding of step (b). Such additional component can be a flavoring component as described above. A flavoring component can impart to the color-fast edible composition a meat-like flavor, a fish-like flavor, a pork-like flavor, a chicken-like flavor, a turkey-like flavor, a shellfish-like flavor or the like. A flavoring component can be selected based on the intended use of the color-fast extruded edible composition. For example, for producing a beef analog, a beef flavoring component can be used. Similarly, for producing a seafood analog, a seafood flavoring component can be used. For producing a pork-analog, a pork flavoring component can be used and for producing a poultry analog (e.g., chicken or turkey), a poultry flavoring component can be used.

### Extrusion Methods of the Present Disclosure

The present disclosure provides an extrusion method of making a color-fast extruded edible composition. The method comprises extruding a mixture comprising an exogenous protein component and pigmented algae cells so as to physically associate the pigmented algae cells with the exogenous protein component to confer a pigmentation to the exogenous protein component, wherein the pigmentation is substantially non-dissociable from the exogenous protein component, thereby making the color-fast extruded edible composition.

As used herein, “extrusion,” “extruding,” and its grammatical variations refer to the process of forcing a mixture through a die of an extruder. When the mixture is blended by the screws of an extruder it is forced through a feeding zone, a mixing zone, and a melting zone before being expelled from the die an extruder, the composition is subjected to a combination of pressure and temperature to physically associate the pigmented algae cells with the exogenous protein to produce a color-fast composition. Typically, conditions sufficient to produce a color-fast composition are created inside the extruder, for example, by screws encased in a barrel of the extruder.

In some cases, extruding comprises heating the mixture to a temperature of at least about 80° C. For example, extruding can comprise heating the mixture to a temperature of at least about 100° C., at least about 110° C., at least about 120° C., at least about 130° C., at least about 140° C., or at least about 150° C. Extruding can also comprise exposing the mixture to steam.

Extruding can comprise heating the mixture under substantially dry conditions. Extruding under substantially dry conditions can comprise extruding with no more than about 20%, no more than about 15%, no more than about 10%, no more than about 5%, no more than about 3%, no more than about 2%, or no more than about 1% humidity. Extruding under substantially dry conditions can also comprise extruding with humidity between 1% and 20%, 2% and 18%, 3% and 17%, 4% and 16%, 5% and 15%, 6% and 14%, 7% and 13%, 8% and 12%, 9% and 11%, or about 10%.

In some cases, the extrusion method generates a color-fast extruded edible composition in a puff form. In some cases, the extrusion method generates a color-fast extruded edible composition that is in a fibrous form. The amount of water present in the mixture of pigmented algae cells and an exogenous protein component can be modified so as to provide the desired form of the color-cast extruded edible composition. In general, a relatively higher water content (e.g., above 40%, particularly, above 45% water) in the mixture of an exogenous protein component and pigmented algae cells can result in a fibrous form of a color-fast extruded edible composition, while a relatively lower water content (e.g. below 40%, particularly, below 35% water) in the mixture of an exogenous protein component and pigmented algae cells can result in a puff form of a color-fast extruded edible composition.

For example, a puff form of a color-cast extruded edible composition can be made by extruding a mixture of pigmented algae cells and exogenous protein that contains less than about 40%, less than about 35%, less than about 30%, less than about 25%, less than about 20%, less than about 15%, less than about 10%, or less than about 5% water, e.g., between: 5% and 40%, 10% and 35%, 15% and 30%, 20% and 30%, and 24% and 26% water. When extruding is performed under these conditions, the mixtures upon exiting from the die of the extruder can produce color-fast extruded edible compositions in puff form. A puff form or a puff protein composition is produced possibly due to the release of pressure and conversion of water into steam and its release from the mixture.

A fibrous form of a color-fast extruded edible composition can be produced by extruding a mixture of pigmented algae cells and exogenous protein containing water at a concentration of more than about 40%, more than about 45%, more than about 50%, more than about 55%, more than about 60%, more than about 65%, or more than about 70%, e.g., between: 40% and 70%, 45% and 65%, 50% and 60%, and 54% and 56%. When extruding is performed under these conditions, the color-fast extruded edible composition in a fibrous form is produced.

Extruding can also comprise pressure cooking the mixture or boiling the mixture. Extruding can also comprise melting and extruding the mixture.

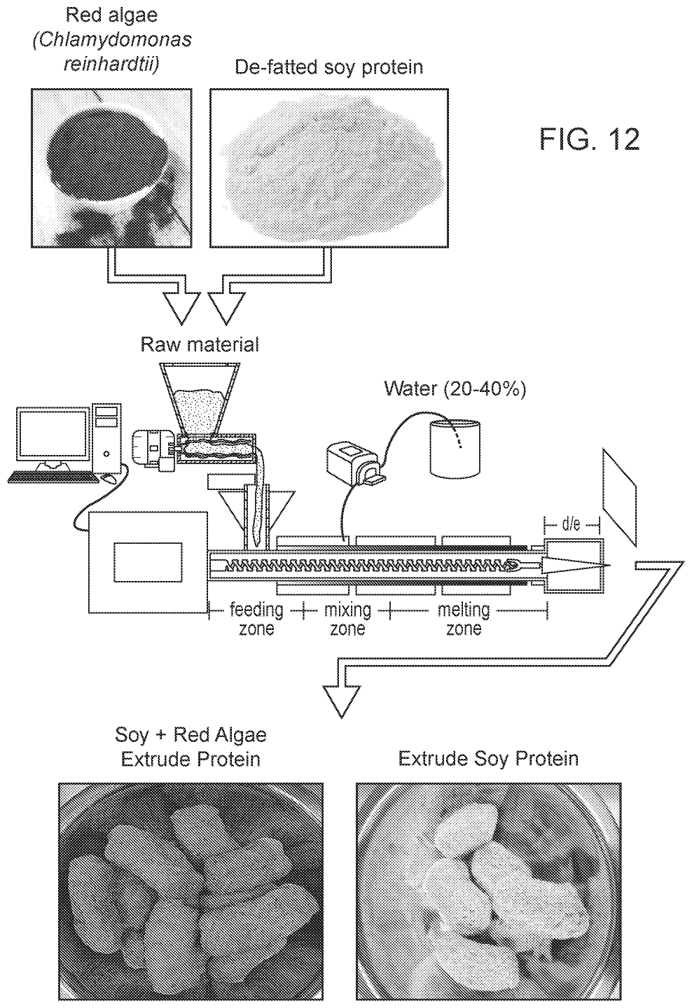

Any suitable extruder available in the art can be used for producing a color-fast extruded edible composition disclosed herein. A typical extruder comprises one or two screws mounted on shafts and that rotate in a barrel. Extruder containing one or two screws provides transport, compression, mixing, cooking, shearing, heating, and shaping of the mixture into color-fast extruded edible composition, for example, puffs or fibrous compositions. An example of an extruder and extrusion method described herein is provided in FIG. 12.

### Color-Fast Extruded Edible Compositions

In some embodiments, the disclosure provides a color-fast extruded edible composition produced by the methods disclosed in herein. A color-fast extruded edible composition so produced comprises an exogenous protein component physically associated with a biomass comprising cells of pigmented algae. The pigmented algae cells can confer to the composition a pigmentation, for example, a red or red-brown pigmentation, that is substantially non-dissociable from the color-fast extruded composition. The color-fast extruded edible composition can be in the puff form or a fibrous form.

As discussed above in connection with the extrusion methods, based on different starting materials and different concentrations of exogenous protein component and pigmented algae cells, different color-fast extruded edible compositions are produced. For example, depending upon the concentration of the pigmented algae cells used in the method of producing, a color-fast extruded edible composition so produced can be used to produce foods, such as a meat analog, a fish analog (e.g., a tuna analog), a pork analog, a chicken analog, a turkey analog, a shellfish analog, or the like. Certain such foods are described below.

### Foods Containing a Color-Fast Extruded Edible Composition

The concentration of the pigmented algae cells can be modified based on the desired use of the color-fast extruded edible composition. Accordingly, the color-fast extruded edible compositions of the present disclosure can be incorporated into a variety of different foods, e.g., meat analog (or meat substitute) (e.g., red meat substitute (e.g. beef substitute), white meat substitute (e.g., pork substitute, avian substitute, e.g., poultry substitute (e.g., chicken, turkey); seafood analogs (e.g., tuna, crab, etc.), and the like.

In certain embodiments, a seafood analog is provided comprising the color-fast extruded edible composition produced by the extrusion methods disclosed herein. In some cases, the seafood analog is a shellfish analog. In some cases, the shellfish analog is selected from the group consisting of: a shrimp analog, a crab analog, a scallop analog, and a lobster analog. In some cases, the seafood analog is a fish analog. In some cases, the fish analog is selected from the group consisting of a tuna analog, a salmon analog, and a cod analog.

In another aspect, a meat analog is provided comprising the color-fast extruded edible composition. In some cases, the meat analog is selected from the group consisting of a beef analog, a turkey analog, a duck analog, an ostrich analog, a dark-meat poultry analog, and a pork analog.

In some cases, a meat analog is a pulled pork analog or barbecue pork analog.

In some cases, the method comprises combining the color-fast extruded edible composition with a liquid. In some cases, the liquid is selected from the group consisting of: a broth, a soup, a sauce, a gravy, a dressing, and a glaze. In some cases, the liquid is selected from the group consisting of: a water and an aqueous salt solution. In some cases, the liquid is selected from the group consisting of: an oil and a fat. In some cases, the method further comprises combining the color-fast extruded edible composition with a solid.

Accordingly, in some embodiments, the disclosure provides a food product comprising the color-fast extruded edible composition produced according to the methods disclosed herein and a second component. The second component can be a liquid selected from the group consisting of: a broth, a gravy, a sauce, a soup, a dressing, and a glaze. The second component can also be a liquid selected from the group consisting of water, an aqueous salt solution, an oil, a fat and any combination thereof. Alternatively, the second component can be a solid selected from the group consisting of a pasta, a noodle, a bread, a bun, a wrapper, a dough, a coating, a grain, a starch, and an icing. In some cases, the food product is selected from the group consisting of a burger, a sandwich, a soup, a dumpling, a stew, a casserole, a wrap, a salad, a biryani, a paella, a jambalaya, and a meatball in sauce.

Notwithstanding the appended claims, the present disclosure is also defined by the following embodiments:

Embodiment 1. An edible composition comprising:
a color-fast mixture comprising an exogenous protein component physically associated with a biomass comprising cells of a Chlamydomonas algae, wherein the exogenous protein component comprises at least about 20% protein (dry weight), such that the biomass confers a red or red-brown pigmentation to the exogenous protein component that is substantially non-dissociable from the exogenous protein component.

Embodiment 2. The edible composition of Embodiment 1, wherein the exogenous protein component comprises a gellable protein.

Embodiment 3. The edible composition of Embodiment 2, wherein the gellable protein is in a gel form and the red or red-brown pigmentation is physically associated with the outside surface of the gel form or as a discontinuous phase trapped within the gel form.

Embodiment 4. The edible composition of Embodiment 2, wherein the gellable protein is in a gel form and is a textured protein.

Embodiment 5. The edible composition of any one of Embodiments 1-4, wherein the color-fast mixture has been heat-treated.

Embodiment 6. The edible composition of any one of Embodiments 1-5, wherein the protein comprises a plant protein.

Embodiment 7. The edible composition of Embodiment 6, wherein the plant protein is selected from the group consisting of: a cereal grain protein, a legume protein, a pulse protein, a tree nut protein, a ground nut protein, a seed protein, a tuber protein, a leaf protein, an algae protein, a microalgae protein, a wheat protein, a soy protein, a nut protein, a pea protein, and any combination thereof.

Embodiment 8. The edible composition of any one of Embodiments 1-7, wherein the protein comprises a fungal protein.

Embodiment 9. The edible composition of Embodiment 8, wherein the fungal protein is selected from the group consisting of: a mushroom protein, a yeast protein, a mycoprotein, and any combination thereof.

Embodiment 10. The edible composition of any one of Embodiments 1-9, wherein the protein comprises an animal protein.

Embodiment 11. The edible composition of Embodiment 10, wherein the animal protein is selected from the group consisting of: a mammalian protein, an avian protein, a fish protein, a seafood protein, and any combination thereof.

Embodiment 12. The edible composition of any one of Embodiments 1-11, wherein the Chlamydomonas algae is of the species Chlamydomonas reinhardtii.

Embodiment 13. The edible composition of any one of Embodiments 1-12, wherein the Chlamydomonas algae comprises a pigment, and wherein the pigment confers the red or red-brown pigmentation to the exogenous protein component.

Embodiment 14. The edible composition of Embodiment 13, wherein the pigment is selected from the group consisting of: heme, protoporphyrin IX, and both heme and protoporphyrin IX.

Embodiment 15. The edible composition of any one of Embodiments 1-14, wherein a substantial proportion of the red or red-brown pigmentation remains associated with the exogenous protein component when the edible composition is in contact with an aqueous solution (e.g., a solution of 1% NaCl at 25° C. for at least 1 minute).

Embodiment 16. The edible composition of Embodiment 15, wherein at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the red or red-brown pigmentation remains associated with the exogenous protein component when the edible composition is in contact with an aqueous solution (e.g., a solution of 1% NaCl at 25° C. for at least 1 minute).

Embodiment 17. The edible composition of any one of Embodiments 1-16, wherein a substantial proportion of the red or red-brown pigmentation does not enter a liquid phase when the edible composition is immersed in a liquid (e.g., when immersed in an oil for at least 1 minute at 25° C.).

Embodiment 18. The edible composition of Embodiment 17, wherein at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the red or red-brown pigmentation does not enter a liquid phase when the edible composition is immersed in a liquid (e.g., when immersed in an oil for at least 1 minute at 25° C.).

Embodiment 19. The edible composition of any one of Embodiments 1-18, wherein a substantial proportion of the red or red-brown pigmentation does not leach or diffuse into a solid or gel phase when the solid or gel phase is in physical proximity (e.g., for at least 1 minute at 25° C.) with the edible composition.

Embodiment 20. The edible composition of Embodiment 19, wherein at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the red or red-brown pigmentation does not leach or diffuse into a solid or gel phase when the solid or gel phase is in physical proximity (e.g., for at least 1 minute at 25° C.) with the edible composition.

Embodiment 21. The edible composition of any one of Embodiments 1-20, wherein the red or red-brown pigmentation is substantially water insoluble (e.g., at 25° C.).

Embodiment 22. The edible composition of Embodiment 21, wherein at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the red or red-brown pigmentation is substantially water insoluble (e.g., at 25° C.).

Embodiment 23. The edible composition of any one of Embodiments 1-22, wherein at least about 0.1%, at least about 0.5%, at least about 1%, at least about 1.5%, at least about 2%, at least about 3%, at least about 4%, or at least about 5% of the edible composition comprises the biomass.

Embodiment 24. The edible composition of any one of Embodiments 1-23, wherein less than about 0.1%, less than about 0.5%, less than about 1%, less than about 1.5%, less than about 2%, less than about 3%, less than about 4%, or less than about 5% of the edible composition comprises the biomass.

Embodiment 25. The edible composition of any one of Embodiments 1-24, further comprising a salt.

Embodiment 26. The edible composition of any one of Embodiments 1-25, wherein the color-fast mixture is comprised of substantially aligned fibers.

Embodiment 27. The edible composition of any one of Embodiments 1-26, wherein the exogenous protein component is comprised of substantially aligned fibers.

Embodiment 28. The edible composition of any one of Embodiments 1-27, further comprising one or more flavorings.

Embodiment 29. The edible composition of Embodiment 28, wherein the one or more flavorings confers a fish-like flavor to the edible composition.

Embodiment 30. The edible composition of any one of Embodiments 1-29, wherein the edible composition has a flake-like shape, a flake-like texture, or both.

Embodiment 31. A seafood analog comprising the edible composition of any one of Embodiments 1-30.

Embodiment 32. The seafood analog of Embodiment 31, wherein the seafood analog is a shellfish analog.

Embodiment 33. The seafood analog of Embodiment 32, wherein the shellfish analog is selected from the group consisting of: a shrimp analog, a crab analog, a scallop analog, and a lobster analog.

Embodiment 34. The seafood analog of Embodiment 31, wherein the seafood analog is a fish analog.

Embodiment 35. The seafood analog of Embodiment 34, wherein the fish analog is selected from the group consisting of a tuna analog, a salmon analog, and a cod analog.

Embodiment 36. A meat analog comprising the edible composition of any one of Embodiments 1-30.

Embodiment 37. The meat analog of Embodiment 36, wherein the meat analog is selected from the group consisting of a beef analog, a turkey analog, a duck analog, an ostrich analog, a dark-meat poultry analog, and a pork analog.

Embodiment 38. A food product, comprising:
(a) a first component comprising an exogenous protein physically associated with pigmented algal cells, in contact with (b) a second component which substantially lacks pigmented algal cells.

Embodiment 39. The food product of Embodiment 38, wherein the second component is selected from the group consisting of: a liquid, a gel, a foam, and a solid.

Embodiment 40. The food product of Embodiment 38 or 39, wherein the exogenous protein comprises a plant protein.

Embodiment 41. The food product of any one of Embodiments 38-40, wherein the plant protein is selected from the group consisting of: a cereal grain protein, a legume protein, a pulse protein, a tree nut protein, a ground nut protein, a seed protein, a tuber protein, a leaf protein, an algae protein, a microalgae protein, a wheat protein, a soy protein, a nut protein, a pea protein, and any combination thereof.

Embodiment 42. The food product of any one of Embodiments 38-41, wherein the exogenous protein comprises a fungal protein.

Embodiment 43. The food product of Embodiment 42, wherein the fungal protein is selected from the group consisting of: a mushroom protein, a yeast protein, a mycoprotein, and any combination thereof.

Embodiment 44. The food product of any one of Embodiments 38-43, wherein the exogenous protein comprises an animal protein.

Embodiment 45. The food product of Embodiment 44, wherein the animal protein is selected from the group consisting of: a mammalian protein, an avian protein, a fish protein, a seafood protein, and any combination thereof.

Embodiment 46. The food product of any one of Embodiments 38-45, wherein the exogenous protein comprises at least about 20%, at least about 25%, at least about 30%, at least about 40%, at least about 50%, at least about 60%, at least about 70%, at least about 80%, at least about 90%, or at least about 95% protein.

Embodiment 47. The food product of any one of Embodiments 38-46, wherein the pigmented algal cells are microalgae cells.

Embodiment 48. The food product of Embodiment 47, wherein the microalgae cells are of the genus Chlamydomonas.

Embodiment 49. The food product of any one of Embodiments 38-48, wherein the pigmented algal cells comprise a pigment that confers a pigmentation to the first component.

Embodiment 50. The food product of Embodiment 49, wherein the pigment is selected from the group consisting of: heme, protoporphyrin IX, and both heme and protoporphyrin IX.

Embodiment 51. The food product of any one of Embodiments 38-50, wherein the pigmented algal cells comprise a mutation in one or more genes in a pathway for chlorophyll synthesis, protoporphyrinogen IX synthesis, or heme synthesis.

Embodiment 52. The food product of Embodiment 51, wherein the pigmented algal cells comprise a mutation in one or more genes selected from the group consisting of: CHLD, CHLI1, CHLI2, CHLH1, ChlB, ChlL, ChlN, protoporphyrinogen IX oxidase, ferrocheletase, protochlorophyllide oxidoreductase, and magnesium chelatase.

Embodiment 53. The food product of Embodiment 51, wherein the pigmented algal cells comprise a mutation in one or more genes that results in a phenotype selected from the group consisting of: upregulated expression of protoporphyrinogen IX oxidase, upregulated expression of ferrocheletase, reduction or elimination of expression of light independent protochlorophyllide oxidoreductase, and a deficiency in the expression of magnesium chelatase.

Embodiment 54. The food product of any one of Embodiments 49, wherein the pigmentation is a red or a red-brown color.

Embodiment 55. The food product of any one of Embodiments 38-54, wherein the food product is a meat substitute or a seafood substitute.

Embodiment 56. The food product of Embodiment 55, wherein the meat substitute is a beef substitute or a pork substitute.

Embodiment 57. The food product of Embodiment 55, wherein the seafood substitute is a shellfish substitute.

Embodiment 58. The food product of Embodiment 57, wherein the shellfish substitute is selected from the group consisting of: a shrimp substitute, a crab substitute, a scallop substitute, and a lobster substitute.

Embodiment 59. The food product of Embodiment 55, wherein the seafood substitute is a fish substitute.

Embodiment 60. The food product of Embodiment 59, wherein the fish substitute is selected from the group consisting of: a tuna substitute, a salmon substitute, and a cod substitute.

Embodiment 61. The food product of any one of Embodiments 38-54, wherein the food product is selected from the group consisting of: a burger, a patty, a hot dog, a meatball, a ground meat, a ground meat product, a meat sauce, a shredded meat, a nugget, a fish ball, a chopped fish, a shredded fish, a fish salad, sushi, sashimi, and a filet.

Embodiment 62. The food product of any one of Embodiments 38-61, wherein the pigmented algal cells comprise at least about 0.1%, at least about 0.5%, at least about 1%, at least about 2%, at least about 3%, at least about 4%, or at least about 5% by weight of the first component.

Embodiment 63. The food product of any one of Embodiments 38-62, wherein the food product further comprises a salt.

Embodiment 64. The food product of any one of Embodiments 38-63, wherein the second component comprises a liquid selected from the group consisting of: a broth, a gravy, a sauce, a soup, a dressing, and a glaze.

Embodiment 65. The food product of any one of Embodiments 38-64, wherein the second component comprises a liquid selected from the group consisting of: water and an aqueous salt solution.

Embodiment 66. The food product of any one of Embodiments 38-65, wherein the second component comprises a liquid selected from the group consisting of: an oil and a fat.

Embodiment 67. The food product of any one of Embodiments 38-66, wherein the second component is a solid selected from the group consisting of: a pasta, a noodle, a bread, a bun, a wrapper, a dough, a coating, a grain, a starch, and an icing.

Embodiment 68. The food product of any one of Embodiments 38-67, wherein the second component is a gel selected from the group consisting of: a mousse, a gummy, a cooked egg, a cake, a bread, a custard, a pie filling, and a flan.

Embodiment 69. The food product of any one of Embodiments 38-68, wherein the food product is selected from the group consisting of: a burger, a sandwich, a soup, a dumpling, a stew, a casserole, a wrap, a salad, a biryani, a paella, a jambalaya, and a meatball in sauce.

Embodiment 70. A method for making a color-fast edible composition, the method comprising:
(a) combining an exogenous protein component comprising at least about 20% protein (dry weight), with a biomass comprising cells of a pigmented algae to produce a mixture;
(b) heating the mixture thereby physically associating the cells of the pigmented algae with the exogenous protein component such that the cells of the pigmented algae confer a pigmentation to the exogenous protein component and the pigmentation is substantially non-dissociable from the exogenous protein component, thereby making the color-fast edible composition.

Embodiment 71. The method of Embodiment 70, further comprising combining the mixture of (a) with at least one additional component prior to or after the heating of (b).

Embodiment 72. The method of Embodiment 71, wherein the at least one additional component is a flavoring component.

Embodiment 73. The method of Embodiment 72, wherein the flavoring component imparts a meat-like flavor, a fish-like flavor, or a shellfish-like flavor to the color-fast edible composition.

Embodiment 74. The method of any one of Embodiments 70-73, wherein the heating of (b) comprises heating the mixture to a temperature of at least about 80° C.

Embodiment 75. The method of any one of Embodiments 70-74, wherein the heating of (b) comprises heating the mixture to a temperature of at least about 100° C., at least about 110° C., at least about 120° C., at least about 130° C., at least about 140° C., or at least about 150° C.

Embodiment 76. The method of any one of Embodiments 70-75, wherein the heating of (b) comprises exposing the mixture to steam.

Embodiment 77. The method of any one of Embodiments 70-76, wherein the heating of (b) comprises heating the mixture under substantially dry conditions.

Embodiment 78. The method of any of Embodiments 70-77, wherein the heating of (b) comprises heating by extrusion.

Embodiment 79. The method of Embodiment 77, wherein the substantially dry conditions comprise heating the mixture in contact with no more than about 20%, no more than about 15%, no more than about 10%, no more than about 5%, no more than about 3%, no more than about 2%, or no more than about 1% humidity.

Embodiment 80. The method of any one of Embodiments 70-79, wherein the heating of (b) comprises pressure cooking the mixture.

Embodiment 81. The method of any one of Embodiments 70-80, wherein the heating of (b) comprises boiling the mixture.

Embodiment 82. The method of any one of Embodiments 70-81, wherein the exogenous protein component comprises at least about 25%, at least about 30%, at least about 40%, at least about 50%, at least about 60%, at least about 70%, at least about 80%, or at about least 90% protein (dry weight).

Embodiment 83. The method of any one of Embodiments 70-82, further comprising washing the color-fast edible composition with an aqueous solution.

Embodiment 84. The method of any one of Embodiments 70-83, further comprising agitating the color-fast edible composition with an aqueous solution.

Embodiment 85. The method of any one of Embodiments 70-84, further comprising combining the color-fast edible composition with a liquid.

Embodiment 86. The method of Embodiment 85, wherein the liquid is selected from the group consisting of: a broth, a soup, a sauce, a gravy, a dressing, and a glaze.

Embodiment 87. The method of Embodiment 85, wherein the liquid is selected from the group consisting of: a water and an aqueous salt solution.

Embodiment 88. The method of Embodiment 85, wherein the liquid is selected from the group consisting of: an oil and a fat.

Embodiment 89. The method of any one of Embodiments 70-88, further comprising combining the edible composition with a solid.

Embodiment 90. A color-fast extruded edible composition comprising:
an exogenous protein component physically associated with a biomass comprising cells of a Chlamydomonas algae, wherein the biomass confers a red or red-brown pigmentation to the color-fast extruded edible composition that is substantially non-dissociable from the color-fast extruded edible composition.

Embodiment 91. The color-fast extruded edible composition of Embodiment 90, wherein the exogenous protein component comprises at least 1% protein by dry weight of the color-fast extruded edible composition.

Embodiment 92. The color-fast extruded edible composition of Embodiment 90 or Embodiment 91, wherein the color-fast extruded edible composition is a puff or fibrous form.

Embodiment 93. The color-fast extruded edible composition of any one of Embodiments 90 to 92, wherein the exogenous protein component comprises a plant protein.

Embodiment 94. The color-fast extruded edible composition of Embodiment 93, wherein the plant protein is selected from the group consisting of: a cereal grain protein, a legume protein, a pulse protein, a tree nut protein, a ground nut protein, a seed protein, a tuber protein, a leaf protein, an algae protein, a microalgae protein, a wheat protein, a soy protein, a nut protein, a pea protein, and any combination thereof.

Embodiment 95. The color-fast extruded edible composition of any one of Embodiments 90 to 92, wherein the exogenous protein component comprises a fungal protein.

Embodiment 96. The color-fast extruded edible composition of Embodiment 95, wherein the fungal protein is selected from the group consisting of: a mushroom protein, a yeast protein, a mycoprotein, and any combination thereof.

Embodiment 97. The color-fast extruded edible composition of any one of Embodiments 90 to 92, wherein the exogenous protein component comprises an animal protein.

Embodiment 98. The color-fast extruded edible composition of Embodiment 97, wherein the animal protein is selected from the group consisting of: a mammalian protein, an avian protein, a fish protein, a seafood protein, and any combination thereof.

Embodiment 99. The color-fast extruded edible composition of any one of Embodiments 90 to 98, wherein the exogenous protein component comprises a gellable protein.

Embodiment 100. The color-fast extruded edible composition of Embodiment 99, wherein the gellable protein is in a gel form and the red or red-brown pigmentation is physically associated with the outside surface of the gel form or as a discontinuous phase trapped within the gel form.

Embodiment 101. The color-fast extruded edible composition of Embodiment 100, wherein the gellable protein is in a gel form and is a textured protein.

Embodiment 102. The color-fast extruded edible composition of any one of Embodiments 90 to 101, wherein the Chlamydomonas algae is Chlamydomonas reinhardtii.

Embodiment 103. The color-fast extruded edible composition of any one of Embodiments 90 to 102, wherein the red or red-brown pigmentation comprises heme, protoporphyrin IX, or a combination thereof.

Embodiment 104. The color-fast extruded edible composition of any one of Embodiments 90 to 103, wherein cells of the Chlamydomonas algae comprise a mutation in one or more genes in a pathway for chlorophyll synthesis, protoporphyrinogen IX synthesis, or heme synthesis.

Embodiment 105. The color-fast extruded edible composition of any one of Embodiments 90 to 104, wherein cells of the Chlamydomonas algae comprise a mutation in one or more genes selected from the group consisting of: CHLD, CHLI1, CHLI2, CHLH1, ChlB, ChlL, ChlN, protoporphyrinogen IX oxidase, ferrocheletase, protochlorophyllide oxidoreductase, and magnesium chelatase.

Embodiment 106. The edible composition of any one of Embodiments 90 to 103, wherein cells of the Chlamydomonas algae comprise a mutation in one or more genes that results in a phenotype selected from the group consisting of: upregulated expression of protoporphyrinogen IX oxidase, upregulated expression of ferrocheletase, reduction or elimination of expression of light independent protochlorophyllide oxidoreductase, and a deficiency in the expression of magnesium chelatase.

Embodiment 107. The color-fast extruded edible composition of any one of Embodiments 90 to 106, wherein the biomass comprising cells of the Chlamydomonas algae confers the red or red-brown pigmentation to the mixture that is substantially non-dissociable from the color-fast extruded edible composition when the composition is in contact with an aqueous solution at 25° C. for at least 1 minute.

Embodiment 108. The color-fast extruded edible composition of Embodiment 107, wherein the aqueous solution is water.

Embodiment 109. The color-fast extruded edible composition of Embodiment 22, wherein the aqueous solution comprises 1% NaCl.

Embodiment 110. The color-fast extruded edible composition of any one of Embodiments 90 to 109, wherein the red or red-brown pigmentation is substantially water insoluble at 25° C.

Embodiment 111. The color-fast extruded edible composition of any one of Embodiments 90 to 110, wherein the biomass comprising cells of the Chlamydomonas algae confers the red or red-brown pigmentation to the color-fast extruded edible composition and the pigmentation that is substantially non-dissociable from the color-fast extruded edible composition into a second component, wherein the second component is a solid or gel phase in physical proximity with the color-fast extruded edible composition.

Embodiment 112. The color-fast extruded edible composition of any one of Embodiments 90 to 111, wherein the color-fast extruded edible composition comprises the biomass comprising the cells of the Chlamydomonas algae at a concentration from 0.1% to 5% of dry weight of the color-fast extruded edible composition.

Embodiment 113. The color-fast extruded edible composition of any one of Embodiments 90 to 111, wherein the color-fast extruded edible composition comprises the biomass comprising the cells of the Chlamydomonas algae at a concentration of less than 5% of dry weight of the color-fast extruded edible composition.

Embodiment 114. The color-fast extruded edible composition of any one of Embodiments 90 to 113, wherein the exogenous protein component is comprised of substantially aligned fibers.

Embodiment 115. The color-fast extruded edible composition of any one of Embodiments 90 to 114, comprising substantially aligned fibers.

Embodiment 116. The color-fast extruded edible composition of any one of Embodiments 90 to 115, wherein the color-fast extruded edible composition has a flake-like shape, a flake-like texture, or both.

Embodiment 117. The color-fast extruded edible composition of any one of Embodiments 90 to 116, further comprising one or more flavorings.

Embodiment 118. The color-fast extruded edible composition of Embodiment 117, wherein the one or more flavorings confer a fish-like flavor to the color-fast extruded edible composition.

Embodiment 119. The color-fast extruded edible composition of Embodiment 117, wherein the one or more flavorings confer a pork-like or barbecue flavor to the color-fast extruded edible composition.

Embodiment 120. A seafood analog comprising the color-fast extruded edible composition of any one of Embodiments 90 to 118.

Embodiment 121. The seafood analog of Embodiment 120, wherein the seafood analog is a shellfish analog.

Embodiment 122. The seafood analog of Embodiment 121, wherein the shellfish analog is selected from the group consisting of: a shrimp analog, a crab analog, a scallop analog, and a lobster analog.

Embodiment 123. The seafood analog of Embodiment 120, wherein the seafood analog is a fish analog.

Embodiment 124. The seafood analog of Embodiment 123, wherein the fish analog is selected from the group consisting of a tuna analog, a salmon analog, and a cod analog.

Embodiment 125. A meat analog comprising the color-fast extruded edible composition of any one of Embodiments 90 to 117 and 119.

Embodiment 126. The meat analog of Embodiment 125, wherein the meat analog is selected from the group consisting of a beef analog, a turkey analog, a duck analog, an ostrich analog, a dark-meat poultry analog, and a pork analog.

Embodiment 127. The meat analog of Embodiment 125, wherein the meat analog is a pulled pork or barbecue pork analog.

Embodiment 128. A food product, comprising the color-fast extruded edible composition of any one of Embodiments 90 to 119, the seafood analog of any one of Embodiments 120 to 124, or the meat analog of any one of Embodiments 125 to 127.

Embodiment 129. The food product of Embodiment 128, wherein the food product is selected from the group consisting of: a burger, a patty, a hot dog, a meatball, a ground meat, a ground meat product, a meat sauce, a shredded meat, a nugget, a dumpling, a steamed bun, a fish ball, a chopped fish, a shredded fish, a fish salad, sushi, sashimi, and a filet.

Embodiment 130. The food product of Embodiment 128 or 129, further comprising a second component, wherein the second component is not red or red-brown pigmented Chlamydomonas algae, and wherein the red or red-brown pigment from the edible composition does not substantially leach or bleed into the second component.

Embodiment 131. The food product of Embodiment 130, wherein the second component is selected from the group consisting of a liquid, a gel, a foam, a solid, and any combination thereof.

Embodiment 132. The food product of Embodiment 130 or 131, wherein the second component comprises a starch.

Embodiment 133. The food product of Embodiment 130 or 131, wherein the second component comprises a second exogenous protein.

Embodiment 134. The food product of Embodiment 133, wherein the second exogenous protein comprises a plant protein, a fungal protein or an animal protein.

Embodiment 135. The food product of Embodiment 130 or 131, wherein the second component comprises a second Chlamydomonas algae biomass.

Embodiment 136. The food product of any one of Embodiments 130 to 135, wherein the second component comprises a liquid selected from the group consisting of: a broth, a gravy, a sauce, a soup, a dressing, and a glaze.

Embodiment 137. The food product of any one of Embodiments 130 to 135, wherein the second component is water or an aqueous salt solution.

Embodiment 138. The food product of any one of Embodiments 130 to 135, wherein the second component comprises a liquid selected from the group consisting of: an oil and a fat.

Embodiment 139. The food product of any one of Embodiments 130 to 135, wherein the second component is a solid selected from the group consisting of: a pasta, a noodle, a bread, a bun, a wrapper, a dough, a coating, a grain, a starch, and an icing.

Embodiment 140. The food product of any one of Embodiments 130 to 135, wherein the second component is a gel selected from the group consisting of: a mousse, a gummy, a cooked egg, a cake, a bread, a custard, a pie filling, and a flan.

Embodiment 141. The food product of any one of Embodiments 130 to 135, wherein the food product is selected from the group consisting of: a burger, a sandwich, a soup, a dumpling, a stew, a casserole, a wrap, a salad, a biryani, a paella, a jambalaya, and a meatball in sauce.

Embodiment 142. A method for making a color-fast extruded edible composition, the method comprising: extruding a mixture comprising an exogenous protein component and pigmented algae cells so as to physically associate the pigmented algae cells with the exogenous protein component to confer a pigmentation to the exogenous protein component, wherein the pigmentation is substantially non-dissociable from the exogenous protein component, thereby making the color-fast extruded edible composition.

Embodiment 143. The method of Embodiment 142, wherein the exogenous protein component is at a concentration from 1% to 15% protein by dry weight of the color-fast extruded edible composition.

Embodiment 144. The method of Embodiment 142, wherein the exogenous protein component is at a concentration from 20% to 90% protein by dry weight of the color-fast extruded edible composition.

Embodiment 145. The method of any one of Embodiments 142 to 144, wherein the pigmented algae cells are Chlamydomonas algae cells.

Embodiment 146. The method of Embodiment 145, wherein the Chlamydomonas algae cells are red or red-brown in pigmentation.

Embodiment 147. The method of Embodiment 146, wherein the red or red-brown pigmentation comprises heme, protoporphyrin IX, or a combination thereof.

Embodiment 148. The method of any one of Embodiments 145 to 147, wherein cells of the Chlamydomonas algae comprise a mutation in one or more genes in a pathway for chlorophyll synthesis, protoporphyrinogen IX synthesis, or heme synthesis.

Embodiment 149. The method of any one of Embodiments 142 to 148, further comprising combining the mixture of (a) with at least one additional component prior to or after the treatment of step (b).

Embodiment 150. The method of Embodiment 149, wherein the at least one additional component is a flavoring component that imparts to the color-fast extruded edible composition a meat-like flavor, a fish-like flavor, or a shellfish-like flavor.

Embodiment 151. The method of any one of Embodiments 142 to 150, wherein the heating of (b) comprises heating the mixture to a temperature of at least 80° C.

Embodiment 152. The method of Embodiment 151, wherein the heating of (b) comprises heating the mixture to a temperature of at least 100° C.

Embodiment 153. The method of any one of Embodiments 142 to 152, wherein the heating of (b) comprises exposing the mixture to steam.

Embodiment 154. The method of any one of Embodiments 142 to 152, wherein the heating of (b) comprises heating the mixture under substantially dry conditions.

Embodiment 155. The method of Embodiment 154, wherein the substantially dry conditions comprise from 1% to 20% humidity.

Embodiment 156. The method of any one of Embodiments 142 to 152, wherein the heating of (b) comprises pressure cooking or boiling the mixture.

Embodiment 157. The method of any one of Embodiments 142 to 156, wherein the extruding comprises melting and extruding the mixture.

Embodiment 158. The method of any one of Embodiments 142 to 157, further comprising washing or agitating the color-fast extruded edible composition with an aqueous solution.

Embodiment 159. A color-fast extruded edible composition made by the method of any one of Embodiments 142 to 158.

Embodiment 160. A color-fast extruded edible composition made by the method of any one of Embodiments 142 to 158, wherein the color-fast extruded edible composition is in a puff or fibrous form.

Embodiment 161. A seafood analog comprising the color-fast extruded edible composition of Embodiment 159 or 160.

Embodiment 162. The seafood analog of Embodiment 161, wherein the seafood analog is a shellfish analog or a fish analog.

Embodiment 163. The seafood analog of Embodiment 162, wherein the seafood analog is selected from the group consisting of: a shrimp analog, a crab analog, a scallop analog, a lobster analog, a tuna analog, a salmon analog, and a cod analog.

Embodiment 164. A meat analog comprising the color-fast extruded edible composition of Embodiment 159 or 160.

Embodiment 165. The meat analog of Embodiment 164, wherein the meat analog is selected from the group consisting of a beef analog, a turkey analog, a duck analog, an ostrich analog, a dark-meat poultry analog, and a pork analog.

Embodiment 166. A food product comprising the color-fast extruded edible composition of Embodiment 159 or 160 and a second component.

Embodiment 167. The food product of Embodiment 166, wherein the second component comprises a liquid selected from the group consisting of: a broth, a gravy, a sauce, a soup, a dressing, and a glaze.

Embodiment 168. The food product of Embodiment 167, wherein the second component comprises a liquid selected from the group consisting of water, an aqueous salt solution, an oil, a fat and any combination thereof.

Embodiment 169. The food product of Embodiment 167, wherein the second component is a solid selected from the group consisting of a pasta, a noodle, a bread, a bun, a wrapper, a dough, a coating, a grain, a starch, and an icing.

Embodiment 170. The food product of Embodiment 167, wherein the food product is selected from the group consisting of a burger, a sandwich, a soup, a dumpling, a stew, a casserole, a wrap, a salad, a biryani, a paella, a jambalaya, and a meatball in sauce.

Embodiment 171. A color-fast extruded edible composition comprising:
an extruded color-fast mixture comprising a plant protein component physically associated with a biomass comprising cells of a red or red-brown pigmentation Chlamydomonas algae, wherein the extruded color-fast mixture is in a puff form and wherein the red or red-brown pigmentation is substantially non-dissociable from the puff.

Embodiment 172. The color-fast extruded edible composition of Embodiment 171, wherein the plant protein component comprises at least 20% protein by dry weight of the color-fast extruded edible composition.

Embodiment 173. The color-fast extruded edible composition of Embodiment 171 or 172, wherein the biomass comprises from 0.1% to 5% by dry weight of the color-fast extruded edible composition.

Embodiment 174. A color-fast extruded edible composition comprising:
an extruded composition comprising a plant protein component physically associated with a biomass comprising cells of Chlamydomonas algae comprising a red or red-brown pigmentation, wherein the extruded composition comprises substantially aligned fibers, and wherein the red or red-brown pigmentation is substantially non-dissociable from the fibers.

Embodiment 175. The color-fast extruded edible composition of Embodiment 174, wherein the plant protein component comprises at least 20% protein by dry weight of the color-fast extruded edible composition.

Embodiment 176. The color-fast extruded edible composition of Embodiment 174 or 175, wherein the biomass comprises from 0.1% to 5% by dry weight of the color-fast extruded edible composition.

Embodiment 177. The color-fast extruded edible composition of any one of Embodiments 174 to 176, wherein the edible composition is a fish analog.

Embodiment 178. The color-fast extruded edible composition of any one of Embodiments 174 to 176, wherein the edible composition is a pork analog.

The following examples are put forth so as to provide those of ordinary skill in the art with a complete disclosure and description of how to make and use the embodiments, and are not intended to limit the scope of what the inventors regard as their invention, nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used.

### EXAMPLES

### Example 1—Comparison of Heat-Treated Compositions to Non-Heat-Treated Compositions

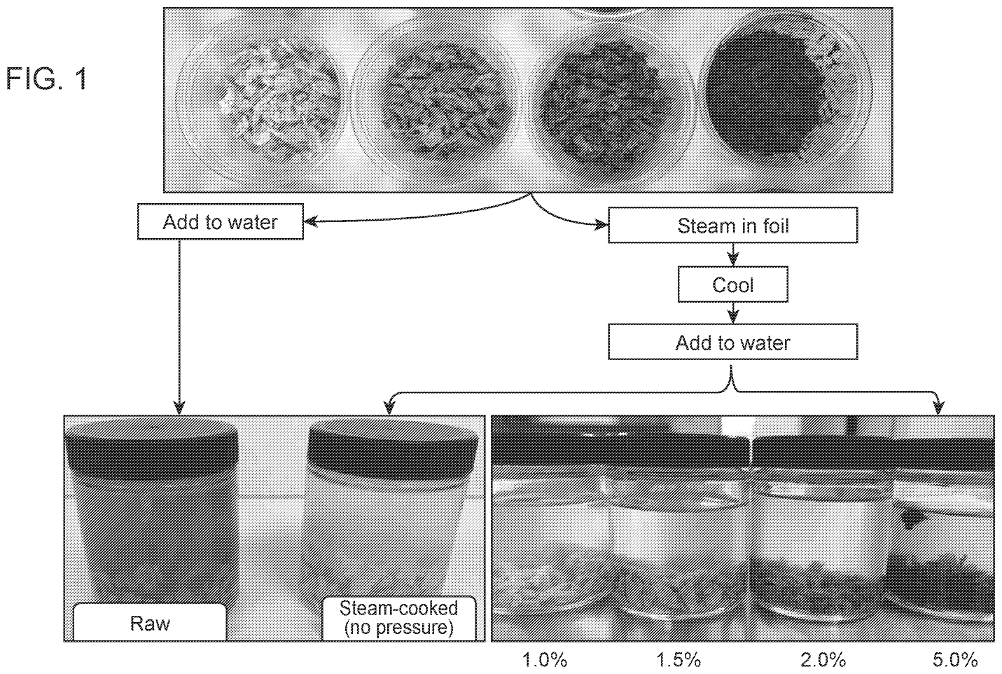

This example demonstrates a method of producing a color-fast edible composition as provided herein. In this example, depicted in FIG. 1, a blend of textured wheat protein was mixed with red algae (at various amounts by weight) and heat treated (steamed in foil). The compositions were cooled and added to water to assess a level of pigment leaching from the textured wheat protein into the water. Non-heat-treated mixtures were used as a control. FIG. 1 demonstrates that there was substantially no or very little pigment leaching into the water for the heat-treated samples, whereas significant pigment leaching was observed with the non-heat-treated samples.

### Example 2—Steam Cooking

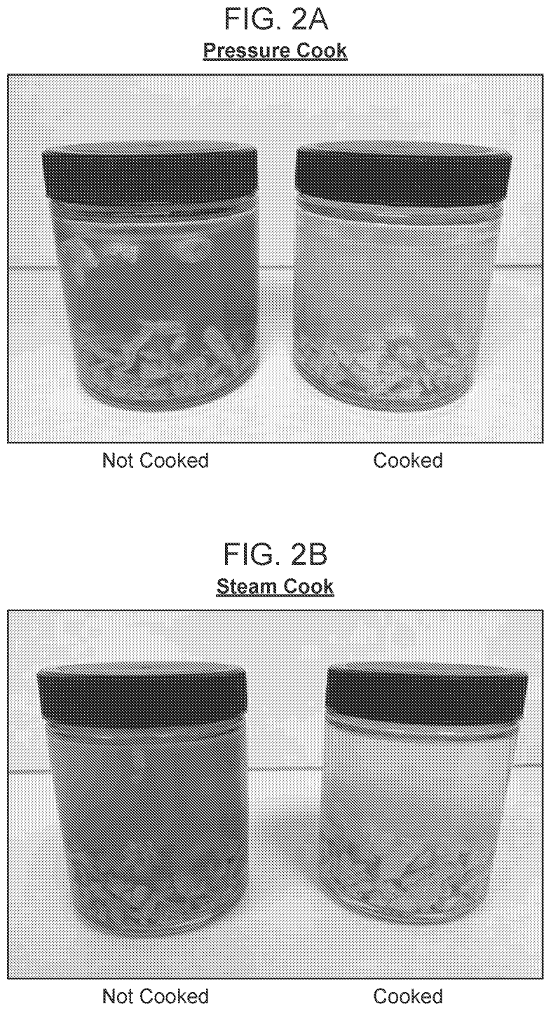

Blends of textured wheat protein, salt, red algae (0.5%), water, and oil were combined. Three samples were made: Sample A—Raw (uncooked, untreated), Sample B—wrapped in foil and pressure cooked (Low pressure Instant Pot, 15 minutes), and Sample C—wrapped in foil and steamed at atmospheric pressure (Stovetop, 15 minutes, no pressure). Samples were then mixed with excess room temperature water and agitated. When Sample A (uncooked, untreated) was dispersed into the water, significant pigment leaching was observed, resulting in a colored liquid. When Sample B (pressure cooked) was added to water, little to no leaching of pigment was observed as compared to the non-pressure-cooked sample (FIG. 2A). When Sample C (steam cooked) was added to water, little to no leaching of pigment was observed as compared to the non-steamed sample (FIG. 2B).

### Example 3—Comparison of Increasing Amounts of Red Algae and Effect on Pigment Immobilization

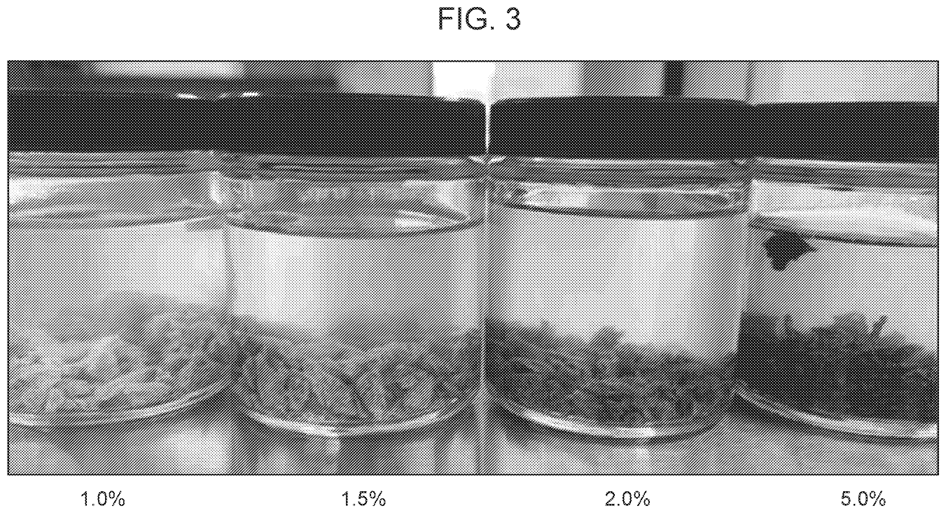

Different samples were prepared with increasing amounts of red algae in the same formula (1.0%, 1.5%, 2.0%, 5.0%) as in Example 2 (minus the oil as it had no effect on the color-fasting). Each sample was steam treated for 15 minutes, then mixed with excess room temperature water and agitated. In all samples, the algae were permanently bound to the surface of the protein as shown in FIG. 3. In the higher concentrations of red algae, there were some red algae/protein clumped particulates that came off with excess agitation.

### Example 4—Washing of Compositions to Determine Pigment Immobilization

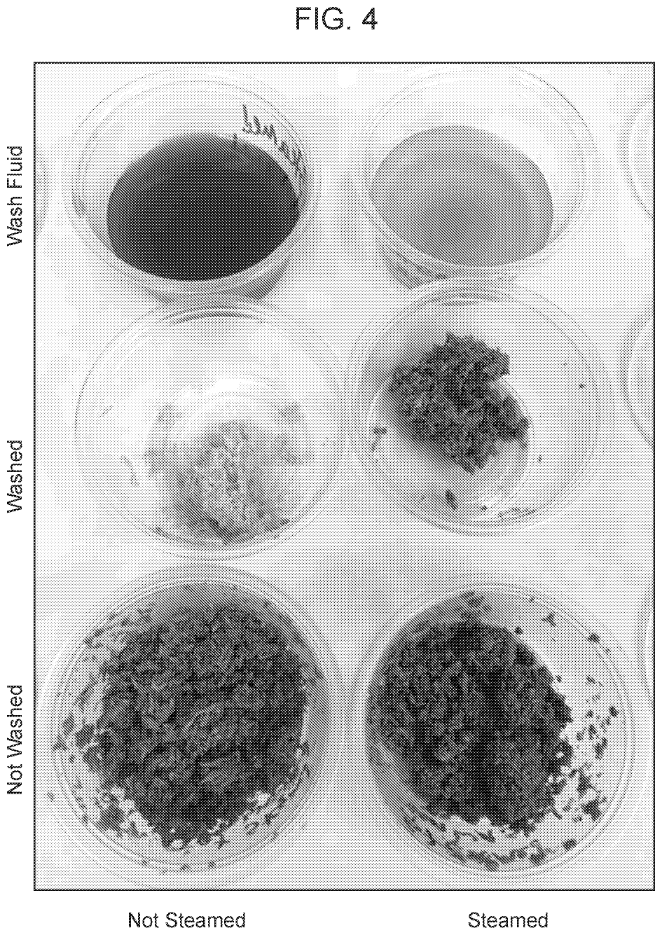

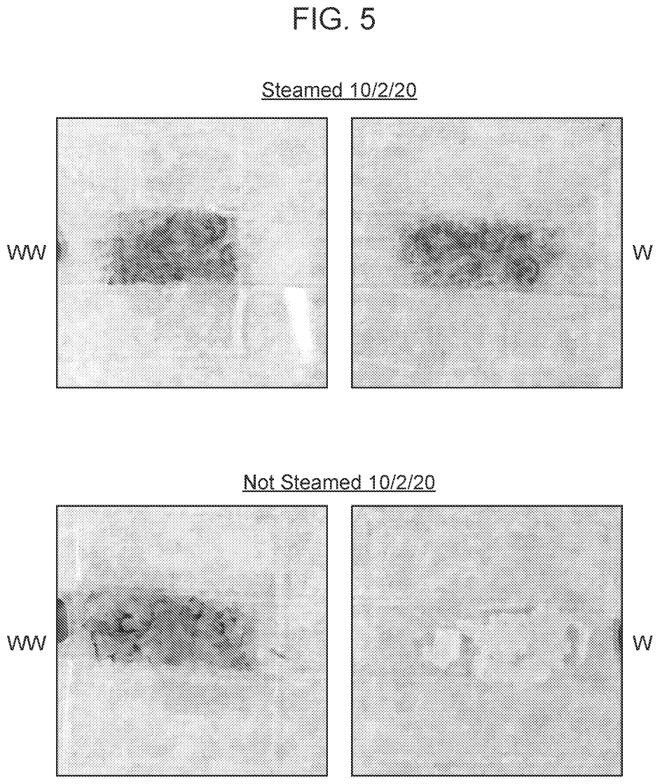

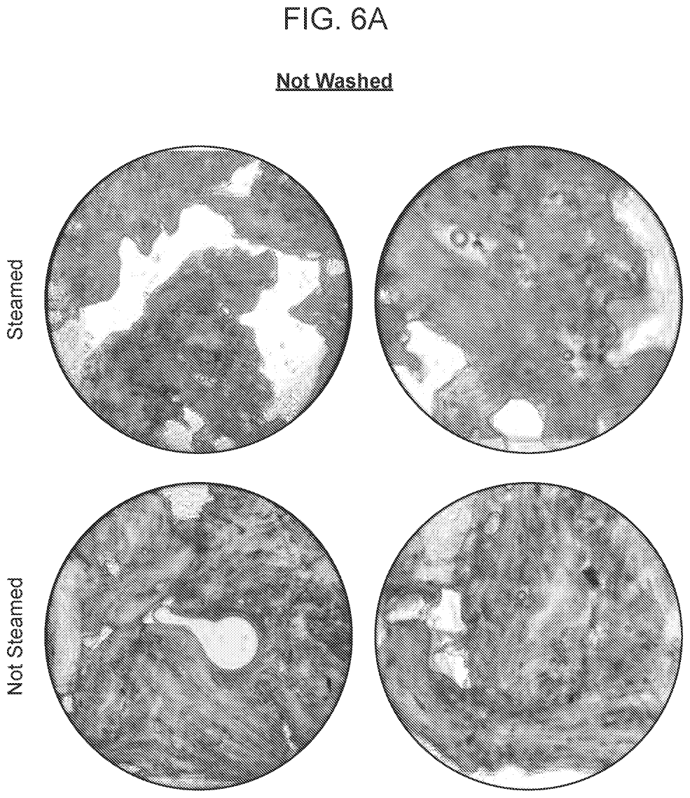

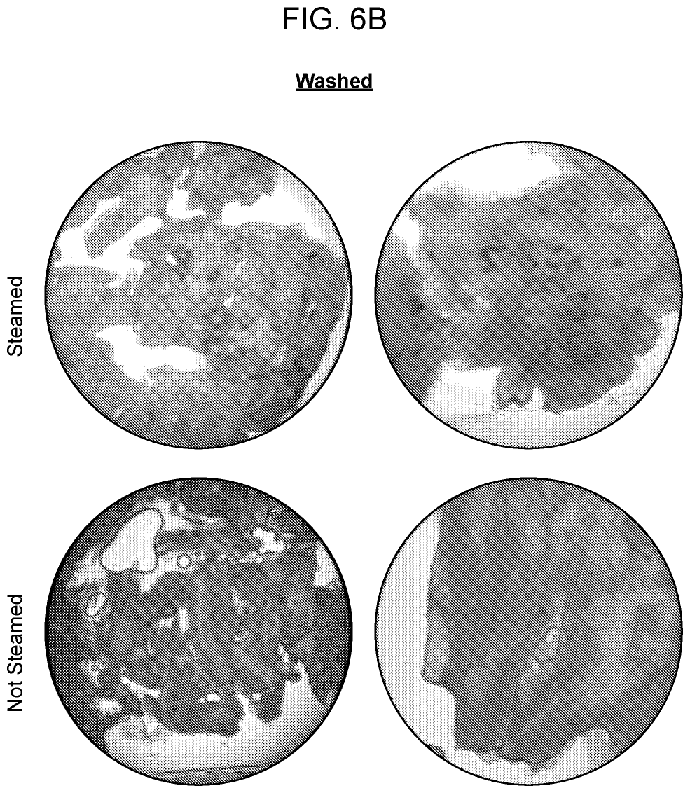

Textured wheat protein fiber was hydrated 1:2 in 1% NaCl and mixed with 2.5% lyophilized red algae powder. A portion of these preparations were sealed in foil, steamed in a pressure cooker for 5 minutes, cooled, and broken up. The steamed material required manually separating fibers, as they stuck together loosely during treatment. 3.0 g each of the steamed and non-steamed materials were then mixed with 20 g 1% NaCl, shaken for one minute, then strained through wire mesh. The wash fluid was retained in a separate container. FIG. 4 shows the steamed and non-steamed fibers, as well as the proportion that was washed, and the wash fluid. Steamed and non-steamed samples were affixed to microscope slides as shown in FIG. 5. Fibers were too large to stay in place under coverslips, so double-sided office tape was used to hold coverslips down and keep sample thickness constant. FIGS. 6A-6B show microscopy images of steamed samples (top row) and non-steamed samples (bottom row). The right panel depicts samples that were washed, while the left panel depicts samples that were not washed. Steamed samples showed larger, denser clumps of red algae, no free-floating single cells. The steamed, washed sample showed clumps of red algae cells around the protein fibers. These images demonstrate that heat treatment immobilizes or physically associates the algal cells with the surface of the protein fiber.

### Example 5—Color-Fasting in Excess Water

Four mixtures were prepared:
1. 1.7% textured wheat protein in 1% NaCl and 0.5% dried Red Algae powder
2. 1.7% textured wheat protein in 1% NaCl (no algae control)
3. 0.5% dried Red Algae powder in 1% NaCl
4. 0.5% Green Algae in 1% NaCl

### Mixture Preparation:

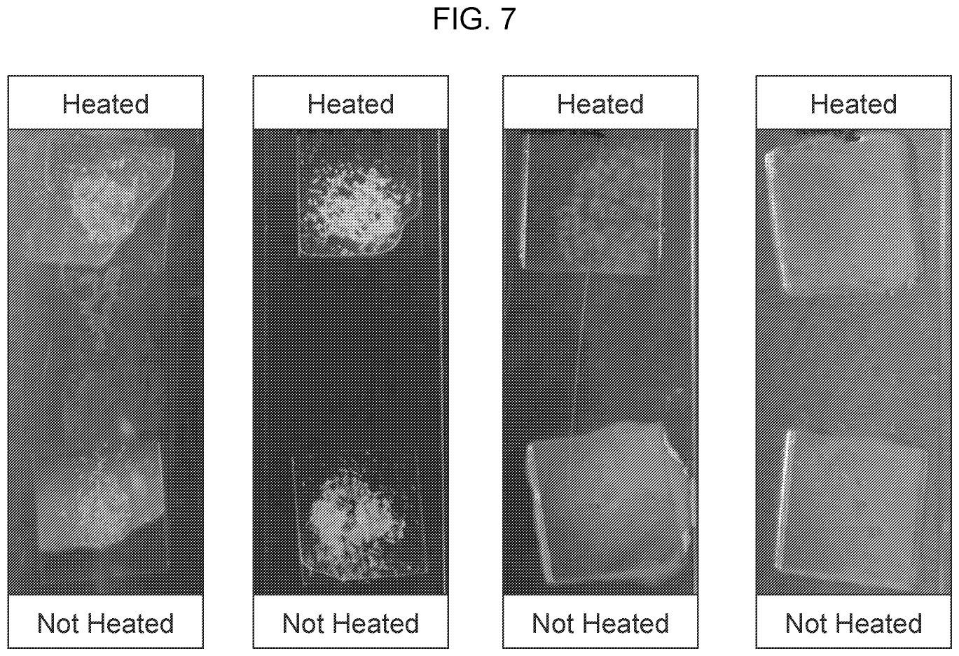

300 ml preparations of mixtures 1-3 were prepared with high shear immersion blender. Spray-dried red algae were used. This created protein fibers small enough for easy microscope slide preparation. 50 ml preparation of spray-dried green algae (mixture 4) was prepared by shaking in a 50 ml centrifuge tube. Each mixture was divided in two. One half from each mixture was heated, and the other was not heated. The heat treatment consisted of transferring the preparation into a beaker, placing it on hot plate, swirling it continuously by hand until 206-212° F., then cooling it. Samples were taken from sediment of cooled and settled preparations, placed on microscope slides, and covered with cover slips as shown in FIG. 7.

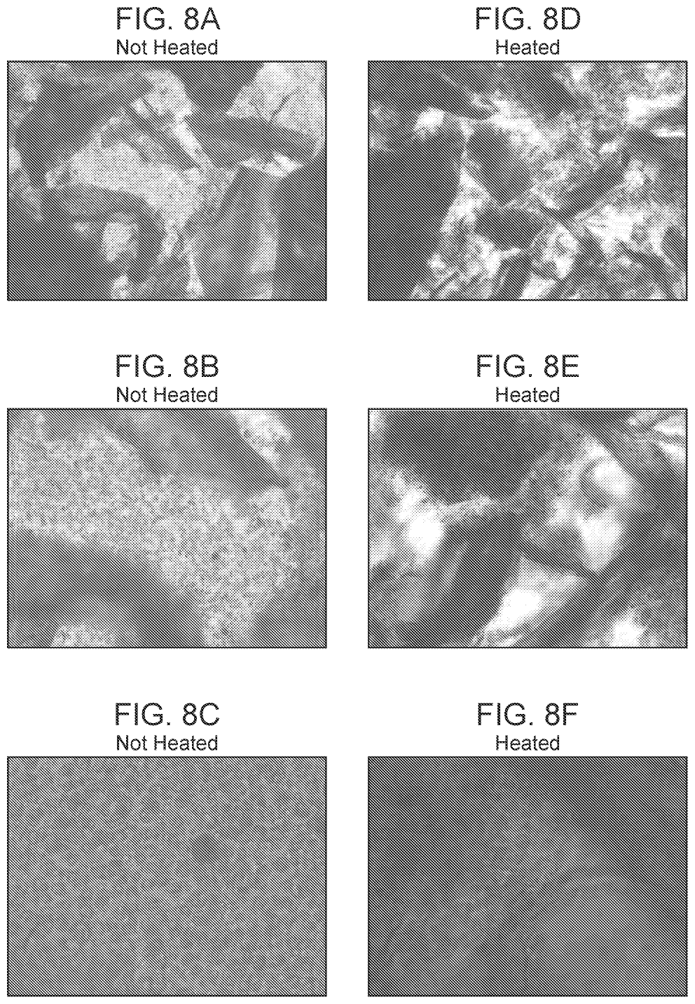

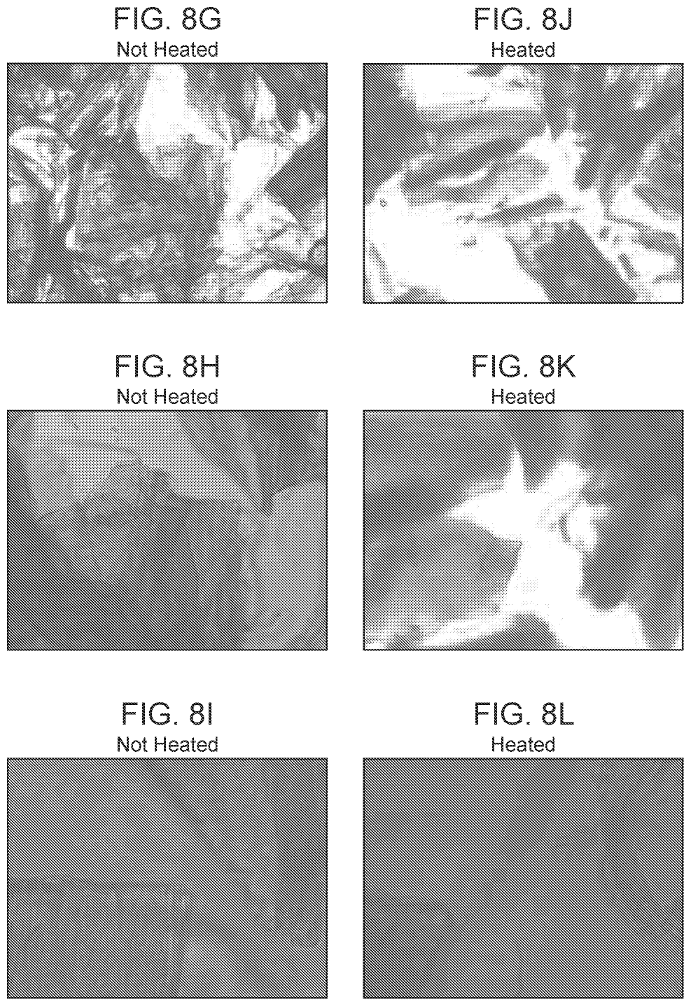

With excess water, protein fibers and red algae clumps appeared more separate than those observed in Example 4. Heated sample sedimented more slowly and loosely (FIGS. 8D-F) than unheated sample (FIGS. 8A-C). A protein-only control was performed to confirm that the reason for clumping was the algae and not the protein. As shown in FIGS. 8G-L, protein alone did not change during heating.

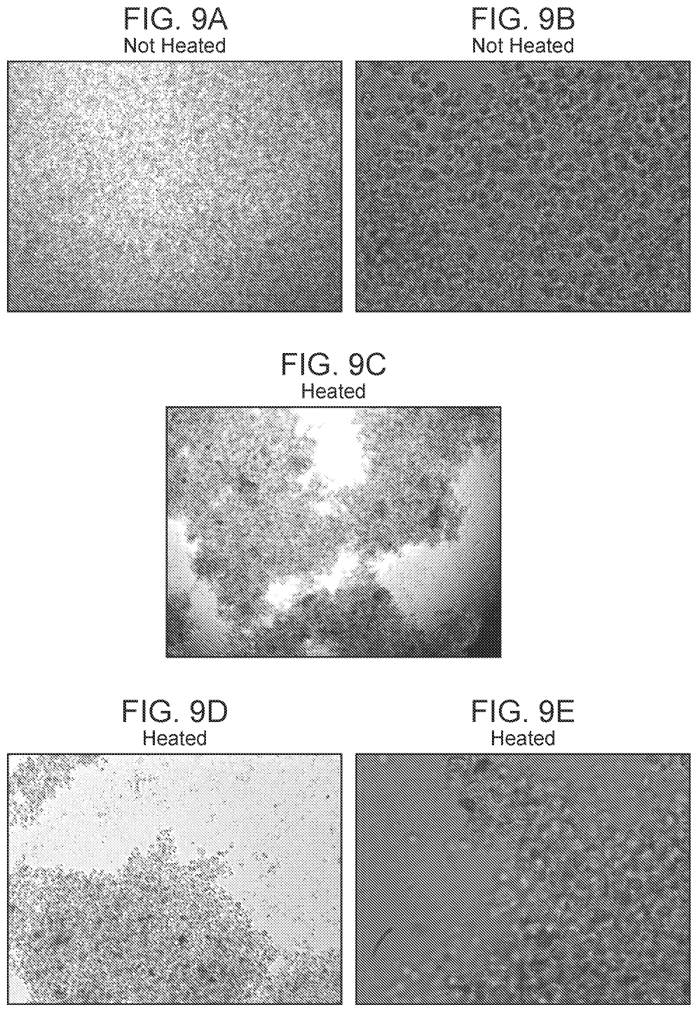

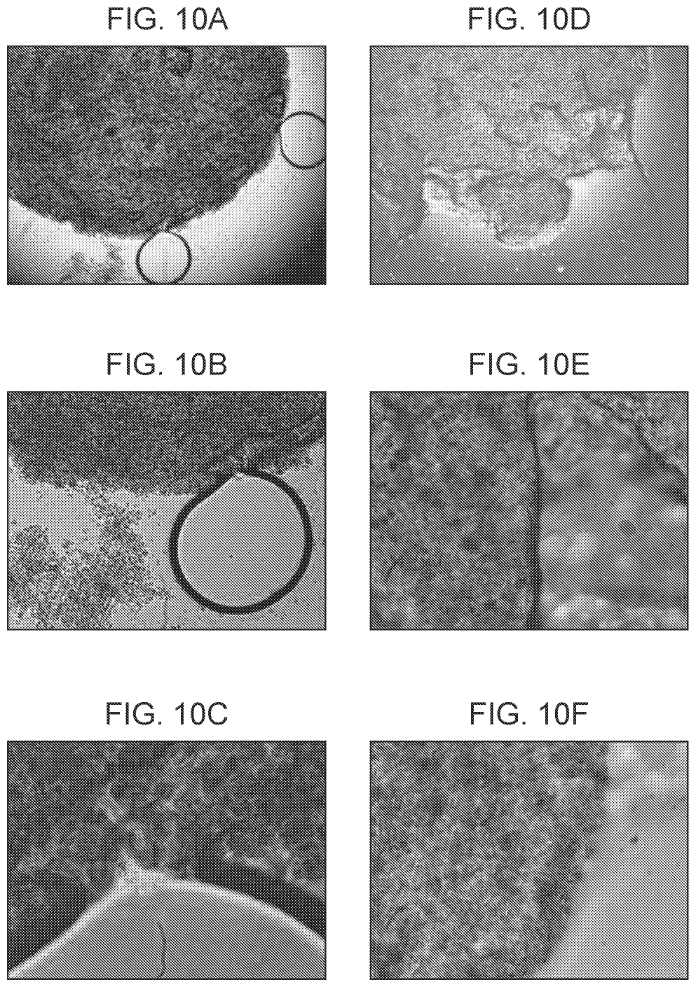

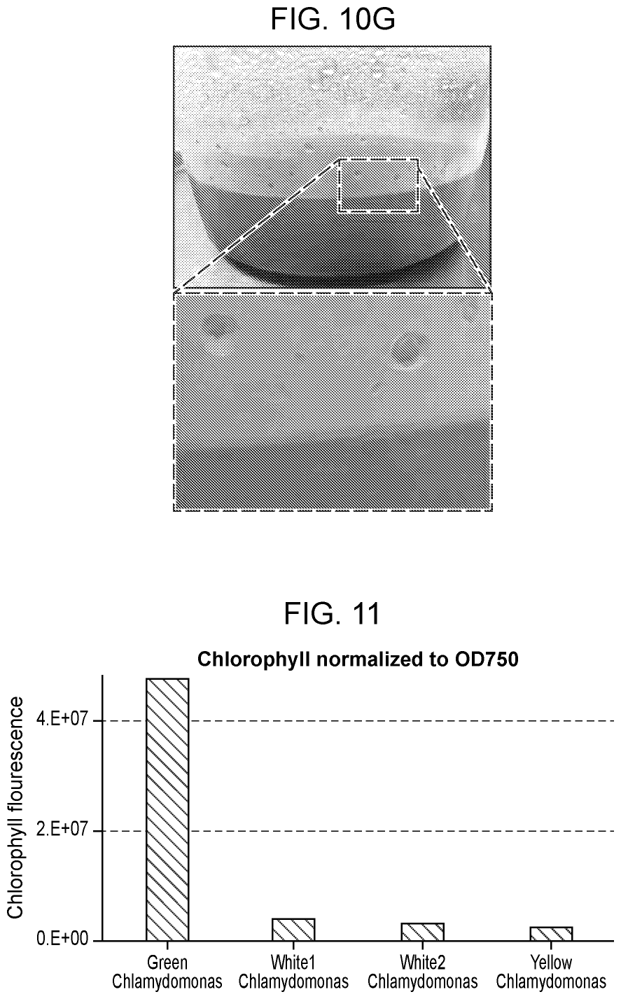

A red algae only control showed that heated red algae solution formed large dense clumps (FIGS. 9C-E). Non-heated red algae solution maintained suspension of individual cells and small cell clusters (FIG. 9A and FIG. 9B). The red algae-only control also formed air bubbles that floated in the heated preparation. The bubbles burst when the coverslip was dropped on the microscope slide. Burst bubbles were imaged successfully (FIGS. 10A-F). The bubbles were stabilized by cohesive sheets of red algae (FIG. 10G).

### Example 6—Flaked-Fish Product

A flaked fish-analog product was prepared with water, wheat protein composition (wheat protein, wheat starch), soy protein isolate, natural flavors, sunflower oil, sea salt, gum Arabic, red algae powder, nature flavor extract (such as containing yeast extract, pea protein isolate, soy lecithin and spice). The ingredients were combined into an edible tuna-like composition. Fibrous, textured wheat proteins were hydrated, then mechanically separated into individual fibers. The mass of individual fibers was combined with the remaining ingredients. The mixture was force extruded to align the individual fibers into loafs. Aligned fiber loafs were steamed for 30 minutes at 80° C., then sliced and flaked after cooling. The flaked composition was portioned into packaged and thermally processed.

Additional fish alternatives are prepared using this method and the ingredients of Table 1.

TABLE 1
Flaked Fish Alterative Compositions

| Ingredient | Flaked fish alternative |
|---|---|
| 1 - Ratio of red algae solids to textured protein solids (by weight, | 0.2-2 g red algae solids to |
| both components on a solids basis) | 100 g textured protein |
| | solids |
| 2 - For red algae containing protoporphyrin IX (PPIX), Ratio of | 0.01-0.1 g PPIX to 100 g |
| PPIX component of algae to textured protein solids (by weight, | textured protein solids |
| both components on a solids basis) | |
| 3 - Ratio of Red Algae solids to hydrated textured or gellable | 0.1-1 g red algae solids to |
| protein component (weight of textured protein solids + weight of | 100 g hydrated textured |
| hydration water) | protein |
| 4 - Percent of red algae solids in final (wet basis) composition | 0.075-0.75% |
| 5 - Percent of textured or gellable protein (solids basis) in the | 15-50% |
| final (wet basis) composition | |
| 6 - Percent of other protein isolate or concentrates (solids basis) in | 0-15% |
| the final (wet basis) composition | |
| 7 - Percent of water in the final (wet basis) composition | 40-80% |
| 8 - Percent of salt (sodium chloride) in the final (wet basis) | 0-2% |
| composition | |
| 9 - Percent of flavoring (as-is ingredient basis) in the final (wet | 0-8% |
| basis) composition | |
| 10 - Percent of Gum Arabic (AKA Acacia Gum, dry basis) in the | 0-5% |
| final (wet basis) composition | |
| 11 - Percent of fat and/or oil in the final (wet basis) composition | 0-10% |
| 12 - Percent of methylcellulose (dry basis) in the final (wet basis) | 0-5% |
| composition | |
| 13 - Percent of Lecithin (dry basis) in the final (wet basis) | 0-2.5% |
| composition | |
| 14 - Percent of a starch (dry basis) in the final (wet basis) | 0-10% |
| composition | |
| 15 - Percent of a flour (dry basis) in the final (wet basis) | 0-20% |
| composition | |
| 16 - Percent of other algae biomass (dry basis) in the final (wet | 0-15% |
| basis) composition | |
| 17 - Percent of a soluble dietary fiber (dry basis) in the final (wet | 0-10% |
| basis) composition of the specified product (same caveat as #4) | |
| 18 - Percent of an insoluble dietary fiber (dry basis) in the final | 0-5% |
| (wet basis) composition of the specified product (same caveat as #4) | |
| 19 - Percent of a gum or gum blend, such as konjac gum or | 0-5% |
| alginate gum (dry basis) in the final (wet basis) composition | |
| 20 - Percent of glycerol (solids basis) in the final (wet basis) | 0-5% |
| composition | |

### Example 7—Comparison of Chlorophyll Content

To compare chlorophyll content for selection of algae biomass, chlorophyll fluorescence was measured and normalized to OD750. The relative amounts of chlorophyll for a green strain, two white strains, and a yellow strain are shown in FIG. 11. To quantitate chlorophyll content in algae for use in color-fasting, strains of interest are measured for chlorophyll fluorescence and normalized to OD750. In some aspects, algae strains are selected having a lower amount of chlorophyll fluorescence as compared to a wildtype or parental green strain.

### Example 8—Formulation of a Dumpling Using Green Algae and Red Algae

To prepare the dumpling wrapper, 6 g Green Algae Flour was whisked into 158 g water. All-purpose flour in the amount of 294 g was kneaded into the slurry until a cohesive dough was formed. The dough was covered and allowed to rest for 30 minutes. Dough was portioned into pieces weighing 13 g, shaped into flattened balls, dusted liberally with flour, and covered. After five minutes, dumpling dough pieces were rolled into thin, flat rounds that were slightly thicker in the center than the outer edges.

Ground pork alternative was prepared by hydrating 200 g textured soy protein in a solution containing 375 g water, 3 g red algae, 3.75 g sea salt, and 1 g pork type flavor. 30 g wheat protein powder and 20 g soy protein powder was mixed into hydrated textured soy protein. The mixture was formed into a loaf, which was then pressure-steamed and cooled. The mixture was extruded through a large sausage die. The extruded log was pressure steamed and cooled. The cooled loaf was forced through a coarse meat grinder. The resulting chopped protein was mixed with 125 g palm shortening, to form the ground pork alternative.

The dumpling filling was prepared by first mixing 6 g sea salt into 565 g finely chopped green cabbage. Then, the cabbage and salt mixture was allowed to stand for 15 minutes, and drained. The drained cabbage was combined with 450 g ground pork alternative with 57 g finely diced fresh ginger, 75 g finely chopped green onions, 33 g miso broth concentrate, 2.75 g white pepper, and salt to taste.

A tablespoon of dumpling filling was placed in the center of a flat dumpling wrapper. Moistened edges of dumpling wrapper were folded over filling and pressed together. Dumplings were placed in a basket and steamed over boiling water for ten minutes.

Visual examination of the dumplings after steaming demonstrated that the color associated with dumpling ground pork alternative, as provided by the red algae, did not bleed into the dumpling wrapper or into the residual water from the steaming process.

### Example 9—Color-Fast Alternative Meat Compositions

Color-fast meat alternatives are prepared with textured or gellable protein and red algae. The protein and algae are combined with water and one or more additional ingredients listed in Table 2 below. In some cases, the textured or gellable protein is first hydrated, and then mechanically separated into individual fibers. The mass of individual fibers was combined with the remaining ingredients. If the alternative meat product is to have aligned fibers, the mixture is extruded to align the individual fibers into loaves. Aligned loaves and/or combined ingredients (for non-aligned fiber products) are heated. In some compositions, the resulting product is sliced, flaked or ground after cooling.

TABLE 2
Alternative Meat Compositions for Color-fasting

| Ingredient | Color-fasted meat alternative |
|---|---|
| 1 - Ratio of red algae solids to textured or | 0.05-3 g of red algae solids |
| gellable protein solids (by weight, both | per 100 g of textured or |
| components on a solids basis) | gellable protein solids |
| 2 - If algae contributing PPIX, Ratio of | 0.0025-0.15 g PPIX to 100 g |
| PPIX component of algae to textured | textured or gellable |
| protein solids (by weight, both components | protein solids |
| on a solids basis) | |
| 3 - Ratio of Red Algae solids to hydrated | 0.02-2 g red algae solids |
| textured protein component (weight of | to 100 g hydrated |
| textured protein solids + weight of hydration | textured or gellable |
| water) | protein |
| 4 - Percent of red algae solids in final (wet | 0.01-2% red algae |
| basis) composition of the specified meat | solids in total meat |
| alternative material | alternative composition |
| 5 - Percent of textured or gellable protein | 10-60% |
| (solids basis) in the final (wet basis) | |
| composition | |
| 6 - Percent of other protein isolate or | 0%-60% |
| concentrates (solids basis) in the final (wet | |
| basis) composition | |
| 7 - Percent of water in the final (wet basis) | 20-90% |
| composition | |
| 8 - Percent of salt (sodium chloride) in the | 0-3% |
| final (wet basis) composition | |
| 9 - Percent of flavoring (as-is ingredient | 1-15% |
| basis) in the final (wet basis) composition | |
| 10 - Percent of Gum Arabic (AKA Acacia | 0-10% |
| Gum, dry basis) in the final (wet basis) | |
| composition | |
| 11 - Percent of fat and/or oil in the final | 0-50% |
| (wet basis) composition | |
| 12 - Percent of methylcellulose (dry basis) | 0-10% |
| in the final (wet basis) composition | |
| 13 - Percent of Lecithin (dry basis) in the | 0-3% |
| final (wet basis) composition | |
| 14 - Percent of a starch (dry basis) in the | 0-60% |
| final (wet basis) composition | |
| 15 - Percent of a flour (dry basis) in the | 0-60% |
| final (wet basis) composition | |
| 16 - Percent of other algae biomass (dry | 0-60% |
| basis) in the final (wet basis) composition | |
| 17 - Percent of a soluble dietary fiber (dry | 0-20% |
| basis) in the final (wet basis) composition | |
| 18 - Percent of an insoluble dietary fiber | 0-15% |
| (dry basis) in the final (wet basis) | |
| composition | |
| 19 - Percent of a gum or gum blend, such | 0-10% |
| as konjac gum or alginate gum (dry basis) | |
| in the final (wet basis) composition | |
| 20 - Percent of glycerol (solids basis) in the | 0-10% |
| final (wet basis) composition | |
| 21 - Actual percent protein, by weight, of | 30-100% |
| the textured or gellable protein | |
| component (solids basis) | |

### Example 10—Production of Soy and Red Algae Extruded Protein Puffs

De-fatted soy protein was combined with red algae to create soy and red algae containing color-fast extruded protein puffs. (FIG. 12). Soy protein and red algae, namely, Chlamydomonas reinhardtii rich in protoporphyrin IX, were fed into the hopper of an extruder at room temperature and conveyed into the feeding zone. A screw equipped with kneading element mixed the soy protein and algae biomass along with injected water at a temperature below 80° C. in the mixing zone. For low moisture extrusion, 20-40% liquid was injected. The mixture continued along the path of the extruder into the melting zone where physical and chemical changes of the soy protein occurred at a temperature above 130° C. The melted mixture was pushed through a die where the mixture formed a puffed shape under the action of shear stress, pressure drop, and water evaporation.

To examine color-fasting of the composition, the extruded soy and red algae protein puffs containing either 0.6%, 1.2%, or 1.8% red algae were rehydrated in cold, warm, and hot water. All puffs appeared pink when rehydrated. The color did not leach from the protein puffs into the water and the water remained clear after 60 minutes of incubation.

### Example 11—Production of Pulled Pork Alternative

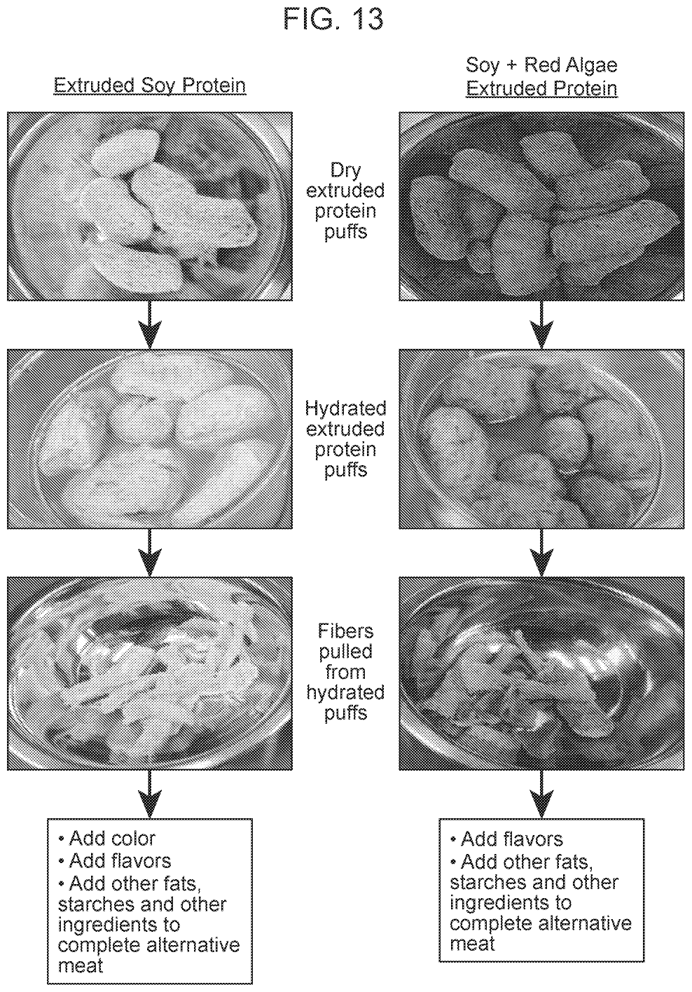

Pulled pork alternative was prepared by hydrating 83 g of extruded textured soy protein puffs with a 1.2% red algae concentration (prepared as described in Example 10) in a solution containing 116 g water, 3 g sea salt, 5 g pork type flavors, and 2 g spices (FIG. 13). The mixture was vacuum sealed in a bag and left to hydrate overnight at refrigeration temperatures. The hydrated mixture was then shredded and combined with 41 g palm oil. The mixture was steamed and cooled to form the pulled pork alternative, then mixed with barbeque sauce.

### Example 12—Comparison of Heat Treated and Non-Heat Treated Flaked Fish Product

A fish analog product was prepared using the recipe shown in Example 6 and formed into loaves. Half of the flaked fish analog product received 30 minute steam treatment at 80° C. and half of the flaked fish analog product did not receive steam treatment. Both steam treated product and non-treated products were cut, flaked and added to water at 23° C. and incubated for 15 minutes. The water containing the steam treated flaked fish analog exhibited a pink-brown color, whereas the water containing the steam treated flaked fish analog was colorless.

### Example 13—Puff Protein Compositions

Color-fast extruded edible compositions in puff form is prepared as disclosed herein and as exemplified in Examples 10 and 11. Following mixtures of exogenous proteins and Chlamydomonas algae cells are extruded to produce puff protein compositions. The protein concentrate is dry powdered composition and contains about 70% protein and other non-protein ingredients, such as carbohydrates, fat, ash, moisture, etc. The non-protein components include: about 15% to 20% carbohydrates, about 2% to 5% fiber, about 1% oil, about 2% to 5% ash, and less than 10% moisture. The red algae powder contains dried Chlamydomonas algae cells.
Mixture 1: 97.0-99.4% vegetable protein concentrate, 0.6%-0.9% red algae powder.
Mixture 2: 97.0-99.4% soy protein concentrate, 0.6%-0.9% red algae powder.
Mixture 3: 97.0-99.4% pea protein concentrate, 0.6%-0.9% red algae powder.
Mixture 4: 97.0-99.4% wheat protein concentrate, 0.6%-0.9% red algae powder.
Mixture 5: 97.0-99.1% vegetable protein concentrate, 0.9%-1.2% red algae powder.
Mixture 6: 97.0-99.1% soy protein concentrate, 0.9%-1.2% red algae powder.
Mixture 7: 97.0-99.1% pea protein concentrate, 0.9%-1.2% red algae powder.
Mixture 8: 97.0-99.1% wheat protein concentrate, 0.9%-1.2% red algae powder.
Mixture 9: 97.0-98.8% vegetable protein concentrate, 1.2%-1.5% red algae powder.
Mixture 10: 97.0-98.8% soy protein concentrate, 1.2%-1.5% red algae powder.
Mixture 11: 97.0-98.8% pea protein concentrate, 1.2%-1.5% red algae powder.
Mixture 12: 97.0-98.8% wheat protein concentrate, 1.2%-1.5% red algae powder.
Mixture 13: 97.0-98.5% vegetable protein concentrate, 1.5%-1.8% red algae powder.
Mixture 14: 97.0-98.5% soy protein concentrate, 1.5%-1.8% red algae powder.
Mixture 15: 97.0-98.5% pea protein concentrate, 1.5%-1.8% red algae powder.
Mixture 16: 97.0-98.5% wheat protein concentrate, 1.5%-1.8% red algae powder.
Mixture 17: 96.0-98.2% vegetable protein concentrate, 1.8%-20.1% red algae powder.
Mixture 18: 96.0-98.2% soy protein concentrate, 1.8%-20.1% red algae powder.
Mixture 19: 96.0-98.2% pea protein concentrate, 1.8%-20.1% red algae powder.
Mixture 20: 96.0-98.2% wheat protein concentrate, 1.8%-20.1% red algae powder.
Mixture 21: 96.0-97.9% vegetable protein concentrate, 2.1%-20.5% red algae powder.
Mixture 22: 96.0-97.9% soy protein concentrate, 2.1%-20.5% red algae powder.
Mixture 23: 96.0-97.9% pea protein concentrate, 2.1%-20.5% red algae powder.
Mixture 24: 96.0-97.9% wheat protein concentrate, 2.1%-20.5% red algae powder.
Mixture 25: 95.0-97.5% vegetable protein concentrate, 2.5%-30.0% red algae powder.
Mixture 26: 95.0-97.5% soy protein concentrate, 2.5%-30.0% red algae powder.
Mixture 27: 95.0-97.5% pea protein concentrate, 2.5%-30.0% red algae powder.
Mixture 28: 95.0-97.5% wheat protein concentrate, 2.5%-30.0% red algae powder.
Mixture 29: 95.0-97.0% vegetable protein concentrate, 3.0%-30.5% red algae powder.
Mixture 30: 95.0-97.0% soy protein concentrate, 3.0%-30.5% red algae powder.
Mixture 31: 95.0-97.0% pea protein concentrate, 3.0%-30.5% red algae powder.
Mixture 32: 95.0-97.0% wheat protein concentrate, 3.0%-30.5% red algae powder.
Mixture 33: 94.5-97.5% vegetable protein concentrate, 3.5%-4% red algae powder.
Mixture 34: 94.5-97.5% soy protein concentrate, 3.5%-4% red algae powder.
Mixture 35: 94.5-97.5% pea protein concentrate, 3.5%-4% red algae powder.
Mixture 36: 94.5-97.5% wheat protein concentrate, 3.5%-4% red algae powder.
Mixture 37: 94.0-96.0% vegetable protein concentrate, 4%-4.5% red algae powder.
Mixture 38: 940.0-96.0% soy protein concentrate, 4%-4.5% red algae powder.
Mixture 39: 940.0-96.0% pea protein concentrate, 4%-4.5% red algae powder.
Mixture 40: 940.0-96.0% wheat protein concentrate, 4%-4.5% red algae powder.
Mixture 41: 93.0-95.5% vegetable protein concentrate, 4.5%-5.0% red algae powder.
Mixture 42: 93.0-95.5% soy protein concentrate, 4.5%-5.0% red algae powder.
Mixture 43: 93.0-95.5% pea protein concentrate, 4.5%-5.0% red algae powder.
Mixture 44: 930.0-950.5% wheat protein concentrate, 4.5%-5.0% red algae powder.

In the above described mixtures, the Mixtures can be used to make meat analogs, such as by hydrating the Mixtures, and combining them with additional components such as one or more of water, salt, flavors, spices and oil. For example: Mixtures 1 to 16 are used to produce color-fast extruded edible compositions that can be used as a poultry analog, such as a chicken analog; Mixtures 5 to 28 are used to produce color-fast extruded edible compositions that can be used as a pork analog; Mixtures 13 to 24 are used to produce color-fast extruded edible compositions that can be used as a fish analog, such as a tuna analog; and Mixtures 29 to 44 are used to produce color-fast extruded edible compositions that can be used as a beef analog.

### Example 14—Flaked Fish Compositions

Flaked fish-analog products are prepared according to Example 6. The ingredients are combined into an edible tuna-like composition. Fibrous, textured vegetable protein is hydrated and mechanically separated into individual fibers. The mass of individual fibers is combined with the remaining ingredients. The mixture is force extruded to align the individual fibers into loafs. Aligned fiber loafs are steamed for 30 minutes at 80° C., then sliced and flaked after cooling. The flaked compositions are portioned into packages and thermally processed by placing the packages into a retort that can sterilize the packages at 240° F. at 28 psi for 28 minutes or at 250° F. at 28 psi for 13 minutes. In some cases, the compositions are packaged in a sealed pouch, such as for shelf storage at room temperature.

Certain flaked fish compositions are shown below; these produce a variety of color-fast flaked fish compositions that provide a spectrum of taste and texture.

Composition 1: 20-22% textured vegetable protein, 0.09-0.11% red algae powder, 0.90-3.0% vegetable oil, water and brine, flavors, solids and salt.

Composition 2: 20-22% textured vegetable protein, 0.11-0.13% red algae powder, 0.90-3.0% vegetable oil, water and brine, flavors, solids and salt.

Composition 3: 20-22% textured vegetable protein, 0.13-0.15% red algae powder, 0.90-3.0% vegetable oil, water and brine, flavors, solids and salt.

Composition 4: 20-22% textured vegetable protein, 0.15-0.17% red algae powder, 0.90-3.0% vegetable oil, water and brine, flavors, solids and salt.

Composition 5: 20-22% textured vegetable protein, 0.17-0.19% red algae powder, 0.90-3.0% vegetable oil, water and brine, flavors, solids and salt.

Composition 6: Any one of Embodiments 1-5, further including gum arabic 0.40-0.44%.

Composition 7: Any one of Embodiments 1-5, further including gum arabic 0.44-0.48%.

Composition 8: Any one of Embodiments 1-5, further including gum arabic 0.48-0.52%.

Composition 9: Any one of Embodiments 1-5, further including gum arabic 0.52-0.56%.

Composition 10: Any one of Compositions 1-9, further including water and brine 40-70%.

Composition 11: Any one of Compositions 1-9, further including a flavoring, an acid, or a spice.

Composition 12: Any one of Compositions 1-10, where the textured vegetable protein is a wheat protein.

Composition 13: Any one of Compositions 1-10, where the textured vegetable protein is a soy protein.

Composition 14: Any one of Compositions 1-13, where the vegetable oil is sunflower oil.

Composition 15: Any one of Compositions 1-13, where the vegetable oil is safflower oil.

Composition 16: Any one of Compositions 1-13, further including a salt, such as NaCl or MgCl₂.

Composition 17: 22-24% textured vegetable protein, 0.09-0.11% red algae powder, 0.90-3.0% vegetable oil, water and brine, flavors, solids and salt.

Composition 18: 22-24% textured vegetable protein, 0.11-0.13% red algae powder, 0.90-3.0% vegetable oil, water and brine, flavors, solids and salt.

Composition 19: 22-24% textured vegetable protein, 0.13-0.15% red algae powder, 0.90-3.0% vegetable oil, water and brine, flavors, solids and salt.

Composition 20: 22-24% textured vegetable protein, 0.15-0.17% red algae powder, 0.90-3.0% vegetable oil, water and brine, flavors, solids and salt.

Composition 21: 22-24% textured vegetable protein, 0.17-0.19% red algae powder, 0.90-3.0% vegetable oil, water and brine, flavors, solids and salt.

Composition 22: Any one of Compositions 17-21, further including gum arabic 0.40-0.44%.

Composition 23: Any one of Compositions 17-21, further including gum arabic 0.44-0.48%.

Composition 24: Any one of Compositions 17-21, further including gum arabic 0.48-0.52%.

Composition 25: Any one of Compositions 17-21, further including gum arabic 0.52-0.56%.

Composition 26: Any one of Compositions 17-25, further including water and brine 40-70%.

Composition 27: Any one of Compositions 17-26, further including a flavoring, an acid, or a spice.

Composition 28: Any one of Compositions 17-27, where the textured vegetable protein is a wheat protein.

Composition 29: Any one of Embodiments 17-27, where the textured vegetable protein is a soy protein.

Composition 30: Any one of Embodiments 17-29, where the vegetable oil is sunflower oil.

Composition 31: Any one of Embodiments 17-29, where the vegetable oil is safflower oil.

Composition 32: Any one of Embodiments 17-31, further including a salt, such as NaCl or MgCl₂.

Composition 33: 24-26% textured vegetable protein, 0.09-0.11% red algae powder, 0.90-3.0% vegetable oil, water and brine, flavors, solids and salt.

Composition 34: 24-26% textured vegetable protein, 0.11-0.13% red algae powder, 0.9%-3.0% vegetable oil, water and brine, flavors, solids and salt.

Composition 35: 24-26% textured vegetable protein, 0.13-0.15% red algae powder, 0.9%-3.0% vegetable oil, water and brine, flavors, solids and salt.

Composition 36: 24-26% textured vegetable protein, 0.15-0.17% red algae powder, 0.9%-3.0% vegetable oil, water and brine, flavors, solids and salt.

Composition 37: 24-26% textured vegetable protein, 0.17-0.19% red algae powder, 0.9%-3.0% vegetable oil, water and brine, flavors, solids and salt.

Composition 38: Any one of Compositions 33-37, further including gum arabic 0.40-0.44%.

Composition 39: Any one of Compositions 33-37, further including gum arabic 0.44-0.48%.

Composition 40: Any one of Compositions 33-37, further including gum arabic 0.48-0.52%.

Composition 41: Any one of Compositions 33-37, further including gum arabic 0.52-0.56%.

Composition 42: Any one of Compositions 33-41, further including water and brine 40-70%.

Composition 43: Any one of Compositions 33-42, further including a flavoring, an acid, or a spice.

Composition 44: Any one of Compositions 33-43, where the textured vegetable protein is a wheat protein.

Composition 45: Any one of Compositions 33-43, where the textured vegetable protein is a soy protein.

Composition 46: Any one of Compositions 33-45, where the vegetable oil is sunflower oil.

Composition 47: Any one of Compositions 33-45, where the vegetable oil is safflower oil.

Composition 48: Any one of Compositions 33-47, further including a salt, such as NaCl or MgCl₂.

Composition 49: 26-28% textured vegetable protein, 0.09-0.11% red algae powder, 0.9%-3.0% vegetable oil, water and brine, flavors, solids and salt.

Composition 50: 26-28% textured vegetable protein, 0.11-0.13% red algae powder, 0.9%-3.0% vegetable oil, water and brine, flavors, solids and salt.

Composition 51: 26-28% textured vegetable protein, 0.13-0.15% red algae powder, 0.9%-3.0% vegetable oil, water and brine, flavors, solids and salt

Composition 52: 26-28% textured vegetable protein, 0.15-0.17% red algae powder, 0.9%-3.0% vegetable oil, water and brine, flavors, solids and salt

Composition 53: 26-28% textured vegetable protein, 0.17-0.19% red algae powder, 0.9%-3.0% vegetable oil, water and brine, flavors, solids and salt

Composition 54: Any one of Compositions 49-53, further including gum arabic 0.40-0.44%.

Composition 55: Any one of Compositions 49-53, further including gum arabic 0.44-0.48%.

Composition 56: Any one of Compositions 49-53, further including gum arabic 0.48-0.52%.

Composition 57: Any one of Compositions 49-53, further including gum arabic 0.52-0.56%.

Composition 58: Any one of Compositions 49-57, further including water and brine 40-70%.

Composition 59: Any one of Compositions 49-58, further including a flavoring, an acid, or a spice.

Composition 60: Any one of Compositions 49-59, where the textured vegetable protein is a wheat protein.

Composition 61: Any one of Compositions 49-59, where the textured vegetable protein is a soy protein.

Composition 62: Any one of Compositions 49-61, where the vegetable oil is sunflower oil.

Composition 63: Any one of Compositions 49-61, where the vegetable oil is safflower oil.

Composition 64: Any one of Compositions 49-63, further including a salt, such as NaCl or MgCl₂.

Composition 65: 28-30% textured vegetable protein, 0.09-0.11% red algae powder, 0.9%-3.0% vegetable oil, water and brine, flavors, solids and salt.

Composition 66: 28-30% textured vegetable protein, 0.11-0.13% red algae powder, 0.9%-3.0% vegetable oil, water and brine, flavors, solids and salt.

Composition 67: 28-30% textured vegetable protein, 0.13-0.15% red algae powder, 0.9%-3.0% vegetable oil, water and brine, flavors, solids and salt.

Composition 68: 28-30% textured vegetable protein, 0.15-0.17% red algae powder, 0.9%-3.0% vegetable oil, water and brine, flavors, solids and salt.

Composition 69: 28-30% textured vegetable protein, 0.17-0.19% red algae powder, 0.9%-3.0% vegetable oil, water and brine, flavors, solids and salt.

Composition 70: Any one of Compositions 65-69, further including gum arabic 0.40-0.44%.

Composition 71: Any one of Compositions 65-69, further including gum arabic 0.44-0.48%.

Composition 72: Any one of Compositions 65-69, further including gum arabic 0.48-0.52%.

Composition 73: Any one of Compositions 65-69, further including gum arabic 0.52-0.56%.

Composition 74: Any one of Compositions 65-73, further including water and brine 40-70%.

Composition 75: Any one of Compositions 65-74, further including a flavoring, an acid, or a spice.

Composition 76: Any one of Compositions 65-76, where the textured vegetable protein is a wheat protein.

Composition 77: Any one of Compositions 65-76, where the textured vegetable protein is a soy protein.

Composition 78: Any one of Compositions 65-76, where the vegetable oil is sunflower oil.

Composition 79: Any one of Compositions 65-78, where the vegetable oil is safflower oil.

Composition 80: Any one of Compositions 65-79, further including a salt, such as NaCl or MgCl₂.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed. It is intended that the following claims define the scope of the disclosure and that methods and structures within the scope of these claims and their equivalents be covered thereby.

We claim:

1. An edible composition comprising:
a color-fast mixture comprising an exogenous protein component physically associated with a biomass comprising cells of a Chlamydomonas algae, wherein the exogenous protein component comprises at least about 20% protein (dry weight),
such that the biomass confers a red or red-brown pigmentation to the exogenous protein component that is substantially non-dissociable from the exogenous protein component.

2. The edible composition of claim 1, wherein the color-fast mixture has been heat-treated.

3. The edible composition of claim 1 or claim 2, wherein the protein is a plant protein, a fungal protein, or an animal protein.

4. The edible composition of any one of claims 1-3, wherein the Chlamydomonas algae is of the species Chlamydomonas reinhardtii.

5. The edible composition of any one of claims 1-4, wherein the Chlamydomonas algae comprises a pigment, and wherein the pigment confers the red or red-brown pigmentation to the exogenous protein component.

6. The edible composition of claim 5, wherein at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% of the red or red-brown pigmentation remains associated with the exogenous protein component when the edible composition is in contact with an aqueous solution (e.g., a solution of 1% NaCl at 25° C. for at least 1 minute).

7. The edible composition of any one of claims 1-6, wherein a substantial proportion of the red or red-brown pigmentation does not enter a liquid phase when the edible composition is immersed in a liquid (e.g., when immersed in an oil for at least 1 minute at 25° C.).

8. The edible composition of any one of claims 1-7, wherein at least about 0.1%, at least about 0.5%, at least about 1%, at least about 1.5%, at least about 2%, at least about 3%, at least about 4%, or at least about 5% of the edible composition comprises the biomass.

9. A seafood analog or a meat analog comprising the edible composition of any one of claims 1-8.

10. A food product, comprising the edible composition of any one of claims 1-8 or the seafood analog or meat analog of claim 9.

11. A method for making a color-fast edible composition, the method comprising:
(a) combining an exogenous protein component comprising at least about 20% protein (dry weight), with a biomass comprising cells of a pigmented algae to produce a mixture;
(b) heating the mixture thereby physically associating the cells of the pigmented algae with the exogenous protein component such that the cells of the pigmented algae confer a pigmentation to the exogenous protein component and the pigmentation is substantially non-dissociable from the exogenous protein component,
thereby making the color-fast edible composition.

12. The method of claim 11, wherein the exogenous protein component comprises at least about 25%, at least about 30%, at least about 40%, at least about 50%, at least about 60%, at least about 70%, at least about 80%, or at about least 90% protein (dry weight).

13. A color-fast extruded edible composition comprising:
an exogenous protein component physically associated with a biomass comprising cells of a Chlamydomonas algae, wherein the biomass confers a red or red-brown pigmentation to the color-fast extruded edible composition that is substantially non-dissociable from the color-fast extruded edible composition.

14. The color-fast extruded edible composition of claim 13, wherein the exogenous protein component comprises at least 1% protein by dry weight of the color-fast extruded edible composition.

15. The color-fast extruded edible composition of claim 13 or claim 14, wherein the color-fast extruded edible composition is a puff or fibrous form.

16. The color-fast extruded edible composition of any one of claims 13-15, wherein the exogenous protein component comprises a plant protein, a fungal protein, or an animal protein.

17. The color-fast extruded edible composition of any one of claims 13-16, wherein the color-fast extruded edible composition comprises the biomass comprising the cells of the Chlamydomonas algae at a concentration from 0.1% to 5% of dry weight of the color-fast extruded edible composition.

18. A seafood analog or a meat analog comprising the color-fast extruded edible composition of any one of claims 13 to 17.

19. A method for making a color-fast extruded edible composition, the method comprising:
extruding a mixture comprising an exogenous protein component and pigmented algae cells so as to physically associate the pigmented algae cells with the exogenous protein component to confer a pigmentation to the exogenous protein component,
wherein the pigmentation is substantially non-dissociable from the exogenous protein component, thereby making the color-fast extruded edible composition.

20. The method of claim 19, wherein the exogenous protein component is at a concentration from 1% to 15% protein by dry weight of the color-fast extruded edible composition.

21. The method of claim 19, wherein the exogenous protein component is at a concentration from 20% to 90% protein by dry weight of the color-fast extruded edible composition.

22. A color-fast extruded edible composition comprising:
a plant protein component physically associated with a biomass comprising cells of Chlamydomonas algae containing a red or red-brown pigmentation, wherein the color-fast extruded edible composition is in a puff form and wherein the red or red-brown pigmentation is substantially non-dissociable from the puff.

23. A color-fast extruded edible composition comprising:
a plant protein component physically associated with a biomass comprising cells of Chlamydomonas algae containing a red or red-brown pigmentation, wherein the color-fast extruded edible composition comprises substantially aligned fibers, and wherein the red or red-brown pigmentation is substantially non-dissociable from the fibers.
